(19) United States
(12) Patent Application Publication
(10) Pub. No.: US 2025/0375126 A1
(43) Pub. Date: Dec. 11, 2025

(54) MOVEMENT MONITORING APPARATUSES USING PATTERNED ELASTOMERIC PRESSURE SENSOR

(71) Applicants: The Texas A&M University System, College Station, TX (US); University of Maryland, College Park, MD (US)

(72) Inventors: Soaram Kim, College Station, TX (US); Byungseok Yoo, Brookville, MD (US); Jung Bin Ahn, College Station, TX (US)

(21) Appl. No.: 19/227,207

(22) Filed: Jun. 3, 2025

Related U.S. Application Data
(60) Provisional application No. 63/657,318, filed on Jun. 7, 2024.

(51) Int. Cl. A61B 5/11

(52) U.S. Cl. CPC A61B 5/112; A61B 2562/0247; A61B 2562/164

(57) ABSTRACT

A highly sensitive and reliable pressure sensor has been successfully fabricated by employing eutectic gallium indium liquid metal as the sensing material and EcoFlex 00-30 silicone as the substrate material via a low-cost fabrication process. The combination of durable mechanical properties in substrate and sensing material contributes to the sensor's superior stretchability and flexibility, resulting in an enhanced sensitivity and a low measurement resolution. The sensor's architecture includes, in a single device, a microchannel with two independent sensing channels. The sensor detects applied pressure accurately and distinguishes pressure distribution across a wide area. By leveraging these features, the sensor proves high efficiency in monitoring movements (e.g., gait) at various speeds with a single sensor attached to a moved object (e.g., human foot). This technology helps differentiate between types of movements (e.g., proper and improper walking postures), proving beneficial for clinical and rehabilitation applications requiring the analysis of patterned movement.

### CROSS REFERENCE TO RELATED APPLICATIONS

This application claims priority under 35 U.S.C. § 119 (e) to provisional patent application U.S. Ser. No. 63/657,318, filed Jun. 7, 2024. The provisional patent application is hereby incorporated by reference in its entirety herein, including without limitation: the specification, claims, and abstract, as well as any figures, tables, appendices, or drawings thereof.

### TECHNICAL FIELD

The present disclosure relates generally to a movement monitoring wearable that utilizes a patterned elastomeric pressure sensor and/or corresponding methods of use and manufacture. The apparatus has applications in, at least: the medical, health & fitness, apparel, robotics, consumer product, and biometric technology industries.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

In recent times, the advancement of pressure sensor technology has been driven by the rapid growth of industries such as automotive, aerospace, healthcare, and robotics, which require accurate pressure sensing measurements. Furthermore, there is a growing need to detect a wide range of small to large pressures in bodies by applying wearable and implantable devices. In this regard, various materials and methods have been explored to improve pressure sensing performances, including high accuracy, sensitivity, and durability. For example, high-conductivity materials such as carbon nanotube (CNT) and graphene were adapted as sensing materials for promising piezoelectric or piezoresistive pressure sensors. The reported sensors exhibited exceedingly low detection limits, which can be attributed to the subtle resistance changes of the sensing materials under minor pressure variations. Still, the quantification range was limited to tens of kPa because of the dimensional breakage of the sensors under high pressure. Capacitive-type pressure sensors using polydimethylsiloxane (PDMS) or ionic liquid as sensing materials showed a wide detection range from small to high pressure, as they could easily vary their dimensions under pressure, thereby changing the output capacity. However, to improve the sensing performance, the microstructure of the sensors should be controlled sophisticatedly using highly pricey processes such as the etching and lithography process, resulting in cost-effective issues. Thus, an innovative fabrication process with low-cost and extra-deformable materials is required to fabricate practically applicable pressure sensors.

Recently, liquid-state electronics using liquid metal have emerged as a promising approach for pressure sensors to overcome the limitations of the existing sensors. Eutectic gallium indium (EGaIn) is one of the common liquid metals used in electronic devices as it maintains low viscosity at near room temperature (1.99.10⁻³ Pa·s) and has excellent electrical conductivity and high readability, allowing it to detect pressure from small to large range. To fully utilize these characteristics of EGaIn as a sensing material, choosing a suitable substrate with durability and susceptible deformability under extreme stimuli is crucial to ensure the effective transmission of small external stimuli (the pressure) to the sensing materials. In this context, the deformable elastomers have opted as sensor substrates to substitute the stiff substrate like a silicon wafer. The commercialized elastomers, including Sylgard™ (Dow Chemical Company; Midland, Michigan), Dragonskin™ (Smooth-On, Inc.; Macungie, Pennsylvania), or EcoFlex® (BASF; Florham Park, New Jersey), were commonly used owing to their mechanical stability, chemical inertness, and biocompatibility. Among them, EcoFlex is a preferred choice due to its high flexibility, printability, and resistance to water and tearing. Many researchers have reported achieving soft and delicate pressure sensors incorporating EGaIn into EcoFlex, expecting them to perform synergistically as pressure sensors due to the advantages of their own materials' properties. In general, a curvy-shaped microchannel with a narrow diameter filled with EGaIn is used to fabricate the pressure sensors in order to enhance the sensing performance. However, this process includes complex and expensive techniques such as laser cutting, surface-controlled coating, or various etching processes with lithography that should be avoided to promote scaled-up industrialization. Despite some papers reporting the fabrication of soft matrix/EGaIn-based torsion, strain, and touch sensors via a simple liquid metal injection method and achieving high sensitivity, their applicability is limited due to inadequate microchannel design. They can only detect one stimulus with a single device, which is impractical for real-world applications, necessitating complex data handling methods, incurring higher costs, and requiring the use of several multiplexers when attaching numerous devices to the sensing area. Hence, there is a strong need for a multi-pressure detection method with a single device, while avoiding any discomforts of employing multiple devices.

As one of the streams, at least some of the present inventors previously developed an EGaIn-EcoFlex-based multi-strain sensor, avoiding complex fabrication steps. The microchannels in the sensor were easily prepared by a 3D-printed mold with unique and novel architecture. See Kim et al., “Egaln-Silicone-based highly stretchable and flexible strain sensor for real-time two joint robotic motion monitoring.” Sens Actuators A Phys 2022, 342, 113659; and “Shin et al., “Hand gesture recognition using EGaIn-silicone soft sensors.” Sensors 2021, 9, 3204.” These publications are hereby incorporated by reference in their entireties herein. The microchannels in a single device detected various applied strains simultaneously, showing the possibility of an innovative next-generation multi-strain sensor.

Building upon this remarkable achievement, the present disclosure adopts as an example, the approach of developing two different sensing channels divided into five (5) sections within a single device. This novel design enables comprehensive analysis of pressure, covering both static and dynamic conditions, facilitating the analysis of pressure applied location and continuous monitoring of the center of pressure movement. The sensors were tested as a practical application for a gait monitoring sensor, exhibiting excellent performance: the sensor analyzed a pressure distribution on foot while walking and distinguished certain patterns between correct walking posture and incorrect walking posture, irrespective of the walking speed. Hence, the designed pressure sensors hold significant potential as a platform for large-dimensional sensors capable of multi-functional pressure detection in disabilities and rehabilitation engineering areas by leveraging the cost-effective fabrication method employing readily available materials.

Thus, there exists a need in the art for novel alternative(s) to real-time wearable movement monitoring systems, including but not limited to those that utilize the new architecting microchannel in a pressure sensor described herein.

### SUMMARY

Current technology cannot indicate the part that is shifting the center from the subject's gait. In addition, typical gait monitoring sensors are manufactured by fabricating a single sensor and then combining multiple sensors into an array system to monitor gait, which is more costly and process intensive. However, the sensor technology represents the transition point very well because a single sensor has already several different sensing channels, resulting from the unique sensor patterned design. A single sensor has different sections according to the sensor patterns and is capable of detecting the subject's gait pressure distribution and its shift. Due to the simplicity of the sensor design and elastomer-based substrates, the sensor system is easy to wear. Also, the sensor is prepared by 3D printing technique. Thus, sensors of any size and any structure can be produced very quickly, reducing fabrication costs and processes.

The present disclosure harnesses the power of IoT to craft wearable sensors tailored for patients (e.g., Parkinson Disease (PD)), providing a paradigm shift from traditional supervised settings to self-monitoring anywhere and anytime. This seamless integration with everyday life not only levels the playing field for PD patients but also offers vast data collection opportunities, fostering advanced research and predictive analytics. Beyond just monitoring, it bridges the gap between periodic assessments and continuous care, making the wearable sensor a true digital health companion for PD patients. With real-time insights in real-world settings, patient autonomy and guide informed therapeutic decisions are empowered. Therefore, the present disclosure lays the foundation for an advanced real-time movement monitoring platform and enhances high-impact, multidisciplinary research in biomedical and rehabilitation engineering.

The present disclosure presents a groundbreaking microchannel-based pressure sensor designed to revolutionize pressure distribution analysis in healthcare monitoring. Unlike traditional sensors requiring multiple devices and extensive wiring, the technology utilizes a unique microchannel design integrated within a single device. The sensor's unique microchannel pattern, comprising various sections with differing numbers or ratios of microchannels affiliated to only two different sensing channels, enables precise and comprehensive pressure mapping in real-time. The sensor constructed using soft elastomers and liquid-state conductors by simplified fabrication process leveraging advanced 3D printing techniques, ensures rapid and cost-effective production, comfortable wearability and seamless integration into various healthcare monitoring applications. The sensor's innovative design eliminates the need for cumbersome setups, enhancing user convenience and facilitating widespread commercialization. The capability of the unique microchannel designed pressure sensor provides not only comprehensive insights into pressure distribution but also enables the capture of dynamic movements such as walking, running, or leg shaking, enhancing its versatility and applicability in various healthcare monitoring settings.

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

As previously alluded to, it is preferred the apparatus be safe, cost effective, and durable.

It is a further object, feature, and/or advantage of the present disclosure to provide a state-of-the-art robust pressure sensor with uniquely patterned sensing channel design without use of complex methods or lithography techniques.

It is still yet a further object, feature, and/or advantage of the present disclosure to with the assistance of a 3D printer, prepare the sensor without using highly priced techniques.

It is still yet a further object, feature, and/or advantage of the present disclosure to simultaneously distinguish various applied pressures and pressure distributions. One example configuration, the current sensor has five different sensing channels in a single device.

It is still yet a further object, feature, and/or advantage of the present disclosure to demonstrate outstanding performance as a pressure measurement and monitoring sensor. One example of the sensor shows an exceptional SNR (72 dB), high sensitivity (66.07 MPa⁻¹), and small measurement resolution (56 Pa) with a wide sensing range up to a few MPa, owing to its novel microchannel architecture.

It is still yet a further object, feature, and/or advantage of the present disclosure to advance gait monitoring systems with a multifaceted approach. For example, improved methods to fabricate the sensor and use of data analysis to derive meaningful insights from the collected results can be harmonized to provide a more efficient sensor.

It is still yet a further object, feature, and/or advantage of the present disclosure to combine the advantages of the liquid state of conductors at room temperature with the high deformability of the elastic polymer substrate provides a highly sensitive and flexible pressure sensor with outstanding performance.

It is still yet a further object, feature, and/or advantage of the present disclosure to monitor real-time movements, including but not limited to the monitoring of human gait patterns with various postures.

It is still yet a further object, feature, and/or advantage of the present disclosure to use a simple test setup to record data, demonstrating the device's performance in measuring the various pressure distributions with good accuracy, and highlighting its strong potential in disability and rehabilitation engineering application areas.

It is still yet a further object, feature, and/or advantage of the present disclosure to, through meticulous testing and validation procedures, ensure the sensor's efficacy while continually striving to enhance its performance and address any arising challenges.

The pressure sensors disclosed herein can be used in a wide variety of applications. For example, to achieve industrialization, it is essential to secure cost-effective manufacturing processes and materials, as well as simple analytical methods. The technology described herein adeptly addresses these challenges, offering a solution that is both accessible and efficient. Particularly in healthcare and medical devices, where seamless wearability is paramount for patient comfort, the sensors boast excellent stretchability and utilize silicon-based materials that ensure wearer comfort without compromise. This innovation has significant potential for enhancing physical therapy, sport performance analysis, rehabilitation, and injury prevention efforts. Furthermore, the sensor technology extends its reach to sports equipment, such as insoles for athletes shoes or fitness trackers. By providing users with real-time feedback on their movement mechanics and performance, the technology enhances athletic training and monitoring. Its ability to address diverse needs and offer practical solutions underscores its value across different sectors, marking a significant stride toward industrialization and widespread adoption.

At least one embodiment disclosed herein comprises a distinct aesthetic appearance. Ornamental aspects included in such an embodiment can help capture a consumer's attention and/or identify a source of origin of a product being sold. Said ornamental aspects will not impede functionality of the elastomeric pressure sensor.

Methods can be practiced which facilitate use, manufacture, assembly, maintenance, and repair of an elastomeric pressure sensor which accomplish some or all of the previously stated objectives. The methods are highly unique they are able to determine the pressure of gestures in a more sensitive manner than that which is known in the art. The methods are further unique in that they accept pressure as an input and can estimate a movement or a gesture as an output, as opposed to analyzing the gesture or movement as an input and estimating a pressure as an output.

The elastomeric pressure sensor can be incorporated into systems or kits which accomplish some or all of the previously stated objectives.

According to some aspects of the present disclosure, a system for monitoring movements comprises an elastomeric pressure sensor patterned with a plurality of sensing channels, the plurality of sensing channels being formed with a plurality of microchannels filled with: a conductive liquid; and a substrate material; and a plurality of sections included within each of the plurality of channels. Each of the plurality of sections are distinguishable from one another because of a difference in a number or a ratio of microchannels included therein.

According to some additional aspects of the present disclosure, the number or the ratio of microchannels increases from a first side of a first sensing channel selected from the plurality of sensing channels to a second side of the first sensing channel selected from the plurality of sensing channels.

According to some additional aspects of the present disclosure, the number or the ratio of microchannels decreases from a first side of a second sensing channel selected from the plurality of sensing channels to a second side of the second sensing channel selected from the plurality of sensing channels.

According to some additional aspects of the present disclosure, the plurality of microchannels comprise two microchannels and/or the plurality of sections comprise five sections. The sections can even be arranged in layers and/or can be arranged so as to provide a three-dimensional, layered effect.

According to some additional aspects of the present disclosure, the conductive liquid comprises a liquid metal. The liquid metal can comprise Eutectic gallium-indium (EGaIn).

According to some additional aspects of the present disclosure, the synthetic elastomer comprises a synthetic elastomer. The synthetic elastomer can comprise a silicone mixture.

According to some additional aspects of the present disclosure, the plurality of microchannels further comprise air. During the manufacturing process, the microchannels are not actively filled with air; air remains trapped inside, helping to maintain their structure and prevent collapse. To function as a pressure sensor, the microchannels should later be filled with a soft, conductive material such as liquid metal.

According to some additional aspects of the present disclosure, the elastomeric pressure sensor is included within a wearable object, the elastomeric pressure sensor is implemented in a mat, the elastomeric pressure sensor forms part of a controller, or the elastomeric pressure sensor is attached to a movable object. Wearable objects can include socks, sleeves, gloves, scarfs, belt, etc.

According to some additional aspects of the present disclosure, the system can also include another sensor that collects biometric data that relates to an aspect other than pressure. For example, the additional sensor can include accelerometers, biometric monitors, position sensors, or fluid level sensors among many others. The ability to monitor pressure and other aspects of biological movements may work synergistically together to provide more wholistic picture of potential problems and solutions in a biometric system and/or with regard to methods for monitoring biometric data. More complex systems that have such a wholistic picture may also proactively aid wearers in movements that the wearer is otherwise not capable of performing. This can include, for example, providing the wearer with an actuated increase in force from a robotic or prosthetic component that aids in moving a part of the body.

According to some other aspects of the present disclosure, a method for monitoring a movement, the method comprises analyzing a widthwise and lengthwise distribution in a plurality of sensing channels having a plurality of sections that are distinguishable from one another because of a difference in a number or a ratio of microchannels included therein. The microchannels are filled with a conductive liquid and a substrate material.

According to some additional aspects of the present disclosure, the method further comprises monitoring human gait with said analysis.

According to some additional aspects of the present disclosure, the method further comprises attaching an elastomeric pressure sensor that includes said sensing channels to an object that experiences the movement.

According to some additional aspects of the present disclosure, the method further comprises allowing the object that experiences the movement to apply repeated, patterned pressure to the plurality of sensing channels. For example, an elastomeric pressure sensor can be laid over a keyboard to assess the ergonomics of one's typing.

According to some other aspects of the present disclosure, a multi-sectioned elastomeric pressure sensor comprises a plurality of sensing channels, the plurality of sensing channels being formed with a plurality of microchannels filled with: a conductive liquid; and a substrate material. A plurality of sections in the multi-sectioned elastomeric pressure sensor are distinguishable from one another because of a difference in a number or a ratio of microchannels included therein.

According to some additional aspects of the present disclosure, the number or the ratio of microchannels linearly increase from a first side of a first sensing channel selected from the plurality of sensing channels to a second side of the first sensing channel selected from the plurality of sensing channels.

According to some additional aspects of the present disclosure, the number or the ratio of microchannels linearly decrease from a first side of a second sensing channel selected from the plurality of sensing channels to a second side of the second sensing channel selected from the plurality of sensing channels.

According to some additional aspects of the present disclosure, the plurality of microchannels comprise two microchannels and/or the plurality of sections comprise five sections. The sections can even be arranged in layers and/or can be arranged so as to provide a three-dimensional, layered effect.

According to some additional aspects of the present disclosure, the conductive liquid comprises a liquid metal. The liquid metal can comprise Eutectic gallium-indium (EGaIn).

According to some additional aspects of the present disclosure, the synthetic elastomer can comprise a synthetic elastomer. The synthetic elastomer can comprise a silicone mixture.

According to some additional aspects of the present disclosure, the plurality of microchannels further comprise air.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIGS. 1A-1C show photographic images of an elastomeric pressure sensor with different conditions, including stretched (FIG. 1A) and twisted (FIG. 1B) under 250% strain. The insets show the condition of the sensor before stretching. FIG. 1C shows photographic images of the bending test setup, enlarged active area, and cross sectional of the sensor. The sensor is attached to the 3D printed homemade two-joint structure.

FIG. 2 shows fractional change in resistance with applied strain over the range of 0-250%. The fitted line to the experimental data points is also shown, and the equation represents a parabolic equation y=Aε²+Bε+C, where y is the relative resistance changes and ε is the strain. Error bars indicate standard deviation based on measurements of the sensors.

FIGS. 3A-3B show variation in sensor resistance with time as the strain is applied to sensor1 (FIG. 3A) and sensor2 (FIG. 3B), between 0 and 80% (0 and) 30° strain, over 12 cycles. Magnified single pulses, between 15 and 45 s in FIG. 3A and FIG. 3B, are shown to estimate the response and recovery times, respectively. Each inset shows a plot with a much-magnified x-axis to determine response and recovery times.

FIG. 4A shows a photograph of the proposed strain sensor attached to a human finger. An electronic circuit connects the sensor terminals to the voltage divider circuit to read signals of finger movements from the proposed strain sensor.

FIG. 4B shows a demonstration of a practical application of the proposed sensor by monitoring human finger movement (PIP and MCP joints). The sensors successfully distinguish two different joints simultaneously, and the resistance changes monotonically at different finger angles. The various movements of PIP and MCP joints were monitored by sensors 1 and 2, respectively.

FIG. 4C shows the sensor can monitor vocal cord movement while speaking Hello and swallowing saliva.

FIG. 5 shows a schematic illustration of sensor fabrication. Uncured liquid EcoFlex 00-30 silicone was poured into a 3D-printed plastic mold with two sensing channels. The silicone was then cured, resulting in open microchannels. This silicone mold was wet-bonded to a spin-coated silicone backing layer in an upside-down orientation to seal the open microchannel. Finally, the sensing material, EGaIn alloy, was injected into the previously air-filled microchannel using a syringe-assisted method.

FIG. 6A shows a cross-section image of air-filled microchannels. The inset graph shows the information on the width and height of the channels with 254 and 130 μm, respectively, with standard deviations of 4 μm.

FIG. 6B shows a digital image of a pressure sensor with superior stretchability. The inset image is the original status of the sensor without stretching. The sensor showed mechanical durability under a stretch of up to 250% without any damage.

FIG. 7A shows a schematic illustration of pressure sensor design.

FIG. 7B shows detailed information on sensor dimension.

FIG. 7C shows photographic image of pressure measurement setup with motorized pressure applying system. A custom-built tester applied pressure to the sensor, ranging from 0 to 0.1 MPa.

FIGS. 8A-8C show pressure sensor test results.

FIG. 8A shows time-dependent sensor resistance variation during gradual pressure loading/unloading (up to 0.1 MPa) across five sections (Sections 1, 2, 3, 4, and 5) over 30 s.

FIG. 8B shows a pressure location index from sensing channel 2 is represented as the output resistance ratio R₂/(R₁+R₂). Resistances are taken from the peak resistance point of each section in FIG. 8A. The distinct index values per section align with theoretical predictions, confirming the sensor's ability to accurately identify applied pressure locations.

FIG. 8C shows resistance alterations in individual sections of sensing channel 2 as pressure gradually rises to 0.05 MPa (solid line). The resistance alterations are accompanied by fitted parabolic equations (dotted line) expressed as y=Ax²+Bx+C, where y symbolizes relative resistance changes and x denotes pressure. The sensor's response fitting this parabolic equation emphasizes its extensive and reliable sensing range.

FIGS. 9A-9C show pressure variation and its corresponding resistance changes across the sections (Sections 1, 2, 3, 4, and 5) over a duration of 0-30 seconds.

FIG. 9A shows a pressure (e.g., weight) was applied to each section (Sections 1, 2, 3, 4, and 5) for the initial 15 seconds and released over the subsequent 15 seconds.

FIG. 9B shows pressure location indexes from channels 1 and 2: The output resistance ratios of ΔR₁ (first-type dot) and ΔR₂ (second-type dot) relative to (ΔR₁+ΔR₂) exhibited inverse trends throughout the sections, reflecting the novel sensor design.

FIG. 9C shows pressure location indexes for channels 1 and 2 were obtained at 7.5 (first-type dot), 10 (second-type dot), 15 (third-type dot), and 20 (fourth-type dot) seconds during the pressure application and release cycle. The calculated indexes for both channels closely paralleled the theoretical predictions (fifth-type dot), irrespective of the time or applied pressure, affirming the index's ability to accurately pinpoint pressure locations.

FIG. 10 shows resistance alterations in individual sections of channel 1 as pressure is gradually applied up to 0.05 MPa (solid line). The corresponding fitted parabolic equations (dotted line) follow the equation as y=Ax²+Bx+C, where y symbolizes relative resistance changes and x denotes pressure. The sensor's ability to accurately detect pressure is evident in the parabolic response of channel 1, highlighting its reliability. Conversely, channel 2 (in FIGS. 8A-8C) exhibits a contrasting trend across different sections, indicating the potential for utilizing a pressure location index to determine the point of pressure application.

FIGS. 11A-11D show a Cyclic test result of the sensor under an applied pressure of 0.05 MPa.

FIG. 11A shows a variation in sensor resistance of sensing channel 2 over time, with the specific periods of applied pressure being highlighted.

FIG. 11B shows a zoomed-in view of the single pulses measured between 80 and 160 seconds from FIG. 11A.

FIGS. 11C-11D show a hysteresis loop of R₁ and R₂, respectively, obtained from section 5 under varying applied pressures.

FIG. 12 shows applied weight (or pressure) with regard to the time for 10 test cycles. The custom-built motorized system controlled the weight (or pressure) load consistently.

FIG. 13 shows durability test result of the sensor. The test was conducted with 0.08 MPa pressure loaded to Section 4 for 1000 cycles, where 100 cycles were applied separately ten times. The sensor was tested with a custom-built tester using a stepper motor, which made a tolerance error of the loading (500 μm error) every 100 cycles. This caused the increase in pressure loading, resulting in an increase in resistance value. However, the results under the test in this condition are very reliable for 1000 cycles.

FIGS. 14A-14B show resistance variations of channel 1 and channel 2 under high pressure up to 0.6 MPa, and the resulting pressure location index. The boxes on the top of the figure indicate the pressed areas for each test.

FIG. 14A shows a sensor response when pressure is applied to a localized area (section 4). Even under high pressure, up to 0.6 MPa (5 kgF), the sensor response exhibited reliable resistance variations and pressure location indexes (R₁: 0.67, R₂: 0.33).

FIG. 14B shows a sensor response when pressure was applied across all sections, involving sections 1 to 5 simultaneously, with the loading of up to 0.6 MPa (100 kgF). The reliable testing results (both resistance variation and pressure location indexes of R1 and R2) demonstrate the potential of the sensor as a gait monitoring sensor.

FIGS. 15A-15B show the sensors' extensive sensitivity and ability to accurately discriminate pressure applied location were evidenced in a piano application.

FIG. 15A configures portions of the sensor as piano keys that are mapped to certain notes/sounds.

FIG. 15B shows that pressing the “piano keys” of FIG. 15A will allow for recording/playing music through a laptop.

FIG. 16A configures portions of the sensor a joystick for car control.

FIG. 16B shows that use of the joystick of FIG. 16A allows for control of the object (car) that is displayed on the screen of the laptop.

FIG. 17 shows an electronic circuit diagram for connecting the sensor terminals to the voltage divider circuit to read signals from the pressure sensor. The module presents a real-time pressure sensing circuit employing a Wheatstone bridge configuration with 440 Ohm resistors and a pressure sensor interfaced with an Arduino UNO microcontroller. The circuit monitors the variation in voltage across the pressure sensor pins, which directly correlates to changes in the sensor's resistance due to applied pressure. The Arduino UNO is connected to the laptop's USB COM port via Arduino IDE and serially reads the sensor's voltage by accurately capturing real-time pressure changes at a rate of 100 ms. The analog output of the Arduino spans from 0 V to 5 V in 1023 increments, ensuring precise data acquisition.

FIG. 18 shows a pentagonal diagram for pressure sensor performance and efficiency comparison. The sensor fabricated in this work occupied the complete pentagonal diagram, showing excellent performance compared to the others.

FIG. 19 shows a detailed performance comparison of pressure sensors.

FIGS. 20A-20F show an application of pressure sensor as gait monitoring system.

FIGS. 20A-20D show digital images of sensor and the test set up for gait monitoring.

FIG. 20E shows resistance changes on walking and jogging cycles.

FIG. 20F show resistance changes on different walking speeds on a treadmill with correct walking posture. In the conducted test, a series of experiments were performed to analyze the gait pattern during standing and walking at various speeds ranging from 0 to 3 mph.

FIG. 21A shows an experimental setup for gait monitoring on a treadmill.

FIG. 21B shows walking speed changes with time.

FIGS. 21A-21B collectively show an experiment where a sensor attached to a subject's right foot and connected to the breadboard, and Arduino collected the signal changes in real-time. The walking speed of the subject was maintained for a minute for each speed, increased from 0 to 3 mph in steps of 1 mph, and decreased back to 0 mph at the same rate.

FIG. 22 shows an electronic circuit diagram for connecting the sensor terminals to the voltage divider circuit to read signals from the pressure sensor. The module presents a real-time pressure sensing circuit employing a Wheatstone bridge configuration with 470 Ohm resistors and a pressure sensor interfaced with Arduino MKR WiFi 1010 microcontroller. The circuit monitors the variation in voltage across the pressure sensor pins, which directly correlates to changes in the sensor's resistance due to applied pressure. The Arduino MKR WiFi 1010 reads the sensor's voltage at a rate of 40 Hz (every 25 ms) to accurately capture real-time pressure changes. The analog output of the microcontroller spans from 0 to 3.3 V in 1023 increments, ensuring precise data acquisition. A 5-voltage battery powers the circuit through the VIN port on the Arduino MKR WiFi 1010 board.

FIGS. 23A-23D show the gait monitoring results were obtained through a wireless system.

FIG. 23A shows an experimental setup for the wireless system. The resistance changes were serially read by the Arduino MKR WiFi 1010 microcontroller and wirelessly transferred to a personal computer via IP address and WiFi connection.

FIG. 23B shows a detailed image of the wireless wearable sensor system, where the microcontroller and the sensor attached to the subject's ankle and foot, respectively, and the wireless system attached on the ankle contains Arduino MKR WiFi 1010 and Li—Po batteries.

FIG. 23C shows tested results obtained with different postures (no pressure, standing posture, and casual walking at 0.5 mph speed) via a wireless system. The prepared system with the sensor clearly differentiates the postures. The zoomed-in graph on the right side of FIG. 23C shows the distinguishable pattern of correct walking posture, showing the overturn of R₂ over R₁. The results align well with the wired system results depicted in FIGS. 20A-20F. A portion of the testing video for the wireless wearable sensor system can be seen in FIG. 21A, where resistance variations are displayed in a laptop screen in real-time.

FIGS. 24A-24B show an example for a strain sensor (Liquid Metal, EGaIn). FIG. 24A shows an example of a system that utilizes a gait monitoring sensor, a transmitter, and an EEG sensor. FIG. 24B shows an output for a sensor detection example to help determine whether there is correct posture or incorrect posture.

FIGS. 25A-25D show a mat-type pressure sensor design. FIG. 25A shows a current pressure design with a linear shape. FIG. 25B shows a cross-section view of the mat. FIG. 25C shows a top plan view of the mat. FIG. 25D shows a detailed view of the foot regions of the mat.

FIGS. 26A-26E show further aspects of the mat-type pressure sensor design of FIGS. 25A-25D. FIG. 26A shows a top plan view of the foot regions of the mat. FIG. 26B shows a detailed view of the bottom layer of the mat, where there are ten different sections for each sensor. The microchannel counts from 2˜20. FIG. 26C shows some alternative dimensions for the bottom layer of the mat. FIG. 26D shows a detailed view of the top layer of the mat, where there are five different sections for each sensor. The microchannel counts from 2˜10. FIG. 26E shows some alternative dimensions for the top layer of the mat.

An artisan of ordinary skill in the art need not view, within isolated figure(s), the near infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

### Example Fabrication of a Stretchable and Flexible Strain Sensor and Results Therefore

A first step of a first example fabrication process for the EGaIn-silicone-based strain sensor, a plastic mold for two microchannels (width: 250 μm, thickness: 125 μm) was prepared by a high quality resolution (16 μm) Objet500 Connex2 3D printer (Stratasys, Ltd.) and rigid/durable Vero family photopolymer inks. The spacing between each microchannel was 500 μm, and the total length of the individual microchannels was 160 mm. And then, a liquid EcoFlex 00-30 silicone prepared by mixing two parts of the silicone materials was poured into the plastic mold and cured at room temperature for four hours. After curing, the silicone mold was detached from the plastic mold and bonded to a thin silicone layer (<100 μm), which was prepared by spin-coating with the liquid EcoFlex 00-30 silicone, in order to seal the opened microchannels of cured silicone mold. These layers were fully cured at room temperature for four hours, without additional pressure or heat treatment. After that, the microchannels were filled with an EGaIn alloy while the air was released through an additional syringe needle inserted into the other terminal port. Lastly, 32 AWG enameled copper wires were connected to the terminal ports of the EGaIn microchannel sensors. The microchannels have three electrical wiring terminals designed with a dimension of 2.5 (length)× 1 (width)× 0.625 (thickness) mm to minimize resistance changes due to the applied strain and facilitate the electrical wire insertion.

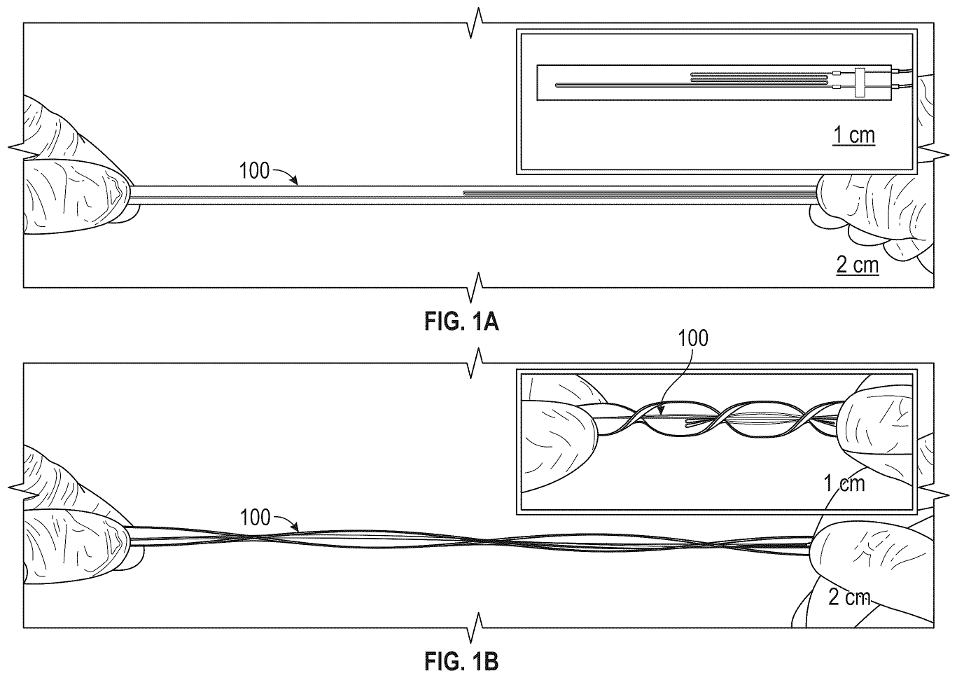

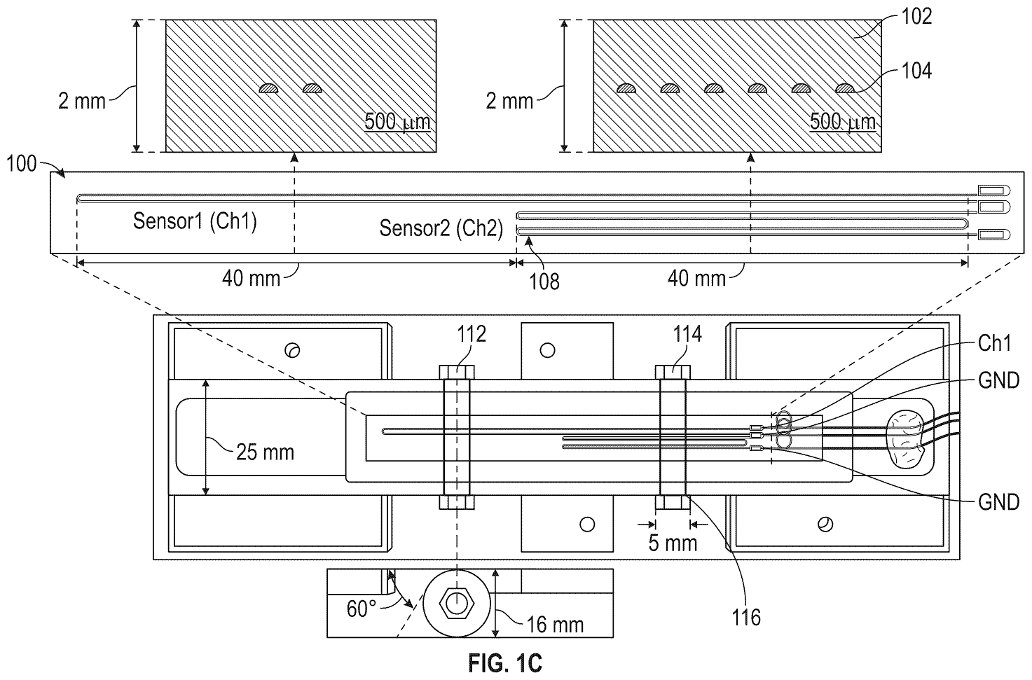

The physical property of EGaIn-Silicone-based strain sensor under different strains was investigated using an X-ray inspection system (North Star Imaging X5000). To obtain the X-ray image, the sensor was attached to a holder prepared by fused deposition modeling 3D printing with polylactic acid filament. The strain sensor was measured by attaching it to a 3D printed homemade two-joint structure, as shown in FIG. 1C, specifically designed to imitate joint movements and hold the strain sensor, and utilizing a data acquisition unit (Gamry 3000 Source/Measure Unit) controlled by a computer to record the data.

The strain sensor 100 comprises the conductive liquid metal (EGaIn alloy), the silicone matrix 102 embedding microchannels 104 (EcoFlex 00-30), and electrical wires. In this sensor design, the size and spacing of each microchannel 104 are the most important parameters because it determines the stretching of the sensor without any damage to the physical structure and leak of EGaIn in the defined region 116 under applied strain that would affect critical device performances. Under the given limitations of commercially available 3D polyjet printer and the specific application (i.e., monitoring of finger movements), the optimal size of the microchannel 104 (250 μm (width)×125 μm (thickness)) was found and the spacing between microchannels 104 was approximately 500 μm. FIGS. 1A-1B show pictures of the strain sensor 100 and its physical stretchability: normal (FIG. 1A), and twisted (FIG. 1B) under the 250% strain. The insets show the conditions of the sensor 100 before stretching. The sensor 100 could be stretched and twisted well without damaging the physical structure. The individual sensing channels 106, 108 (i.e., sensors 1 and 2) have different lengths, such as 80 mm (by two-folds, sensor 1) and 40 mm (by four-folds, sensor 2), as shown in FIG. 1C, while the total channel length of both individual sensing channels is equal to 160 mm. With this novel design, two sensing channels 106, 108 in a single sensor configuration can detect different joint movements quickly and effectively. To investigate the sensor performance with the different joint movements, the sensor was attached to a 3D printed homemade two-joint structure (i.e., bending test fixture) to simulate joint finger movement using the EcoFlex silicone as an adhesive material. The two joint sections 112, 114 (Joint1 and Joint2, respectively) of the bending test fixture pretend to be the proximal interphalangeal (PIP) and metacarpophalangeal (MCP) joints of the fingers. Fabric adhesive tape was also attached to the 3D printed fixture to strengthen the silicone-based bonding layer between the test fixture and the sensor. Note that the joint bending areas located in the boxes that include the two joint sections 112, 114 (i.e., test areas) in FIG. 1C were not bonded to the 3D printed fixture, and the test areas (5 mm) were able to be bent freely as the joint moves. The various movements of joints 1 and 2 112, 114 were monitored by sensors 1 and 2 106, 108, respectively. Additionally, FIG. 1C shows the cross-sectional view of the microchannels, and they have semi-elliptical shapes, although their original design for the silicone molding is a rectangular shape. This is because we selected the Glossy Finish option for a smooth finish of the microchannel pattern surface in the 3D printing system; thus, the edges of the microchannel were rounded due to the increase of the photo resin UV exposure time, resulting in changing the rectangular shape into the semi-elliptical shape.

A measurement setup, including a data acquisition unit (Gamry 3000 Source/Measure Unit) with a laptop, and a 3D printed homemade two-joint structure (i.e., test fixture) were used to apply several different strains to the sensor. To determine the precisely applied strains to the sensor during the experiment, we set up different angles on the two-joint structure, such as 15, 30, 45, 60, 75, and 90°. The output of the sensor with various strains up to 250% (i.e., 90° bend) was measured by the data acquisition unit, which is connected to the electrical wiring terminals of the sensor. The applied strain was adjusted manually using the 3D printed homemade two-joint structure. The applied strain (ε) is defined as ΔL/Lₒ, where ΔL is total elongation, and Lₒ is the original length. Since the attached sensor on the two-joint structure is bent and stretched with different angles (i.e., deformed as a curve shape), the total elongation is equal to arc length. Arc length(S) is defined as rθ, where r is radius and θ is the central angle in radians.

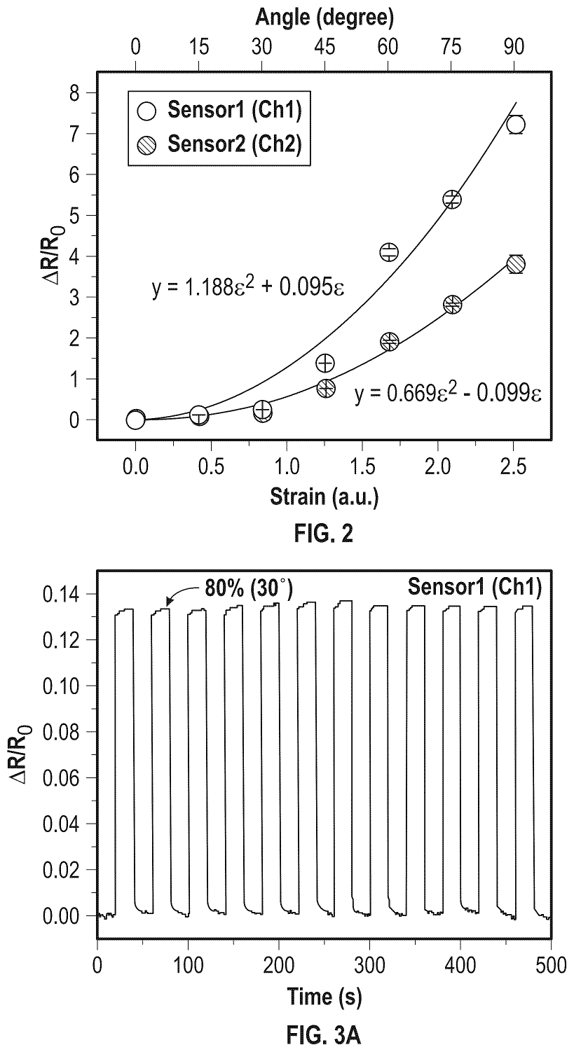

Applied strains with different angles were calculated and used to plot the ΔR/Rₒ (the sensor response) vs ε (strain), as shown in FIG. 2. To confirm the calculated strain with the equation, the strain distributions across the sensor were simulated using COMSOL Multiphysics. The strain sensor was attached with EcoFlex silicone as an adhesive material to the 3D printed homemade two-joint structure except for the test area (5 mm) to apply various strains with different angles to the sensor. Therefore, for simulations, the non-test area of the sensor was assumed to be fixed, and the test area was assumed to be bent at 30° of the sensor. The strain distribution for the sensor, which is responsible for producing relative resistance change in response to strain, increased on the bent area of the sensor. From the strain distribution in the test area of the sensor, the average strains were computed to be 0.8, while the calculated strain with the equation mentioned above was 0.83. Thus, the closeness of the strain values determined from two entirely unrelated methods is remarkable and underlines the reliability of the estimation process.

To investigate the hyper elastic material deformation behavior of the sensor by strains, we performed an X-ray inspection of the strain sensor under three uniaxial strains of 10%, 40%, and 70%. Notably, the filled liquid EGaIn alloy in the microchannels varies its shape according to the silicone deformation induced by the strain applied to the sensor. The liquid metal microchannels retain their shape well due to the surface tension effect of the EGaIn alloy material. The total volume of the EGaIn alloy should be the same before and after strain is applied to the sensor because the EGaIn alloy in the liquid state is incompressible. In addition, it is confirmed that the strain is applied equally to two sensing microchannels with different active sensing lengths of 40 and 80 mm. For example, when Sensor1 (Ch1) is extended 24 mm along its length, Sensor2 (Ch2) is extended about 12 mm, but the strain applied to both microchannels is equal. Ideally, the change in resistance caused by uniaxial strain applied to the sensor should be similar for sensors 1 and 2.

FIG. 2 shows the response of the strain sensor to varying applied strains ranging from 0% to 250% (i.e., 0-90°). The sensor response (ΔR/Rₒ) increases with increasing the strain, and it can fit into a parabolic equation y=Δε2+Bε+C, where y is the relative sensor response and ε is the strain. Because there is no sensor response when there is no applied strain to the sensor (i.e., 0°), the value of C in this equation is zero, as shown in FIG. 2. In addition, the value A in the equation is related to the sensitivity of a sensor, which is generally defined as the relationship coefficient of the output to the input quantity, because the higher value of A yields a more significant sensor response (ΔR/Rₒ), resulting in better sensitivity in the sensor. Thus, the values of A for both sensors from FIG. 2 are 0.669 (sensor 1) and 1.188 (sensor 2), and the value of sensor 2 is about two times higher than that of sensor 1. This is because the sensing channels of sensor 2 were four-folded while that of sensor 1 was two-folded, making their relative resistance changes (i.e., sensor response) about two times higher, although the total length of both individual sensing channels is the same. This difference from the unique and novel architecture makes our strain sensor a strong candidate for practical applications requiring multiple sensors in limited areas, especially joint movements, since these two sensors' results do not overlap. Furthermore, the sensor response (ΔR/Rₒ) for a specific applied strain can be utilized to calculate a gauge factor (GF) of the sensor, which is an important parameter (i.e., sensitivity) to understand the performance of the sensor. The gauge factor can be calculated using the formula GF=(ΔR/Rₒ)/ε, where ΔR/Rₒ is the fractional change in resistance for a specific applied strain (ε) while ΔR is the change in resistance and Rₒ is the original resistance. The GF of sensor 1 was 0.16 at 40% strain (i.e., 15°) and increased to 1.5 at 250% strain (i.e., 90°), while the GF of sensor 2 was 0.28 at 40% strain and increased to 3 at 250% strain. Furthermore, to clarify the repeatability of the sensor, we tested the sensor performance after the extensive response had taken one year apart with the same strain range between 0% and 250% (i.e., 0°-90°) and the sensor shows very similar results on both sensors 1 and 2 compared to FIG. 2.

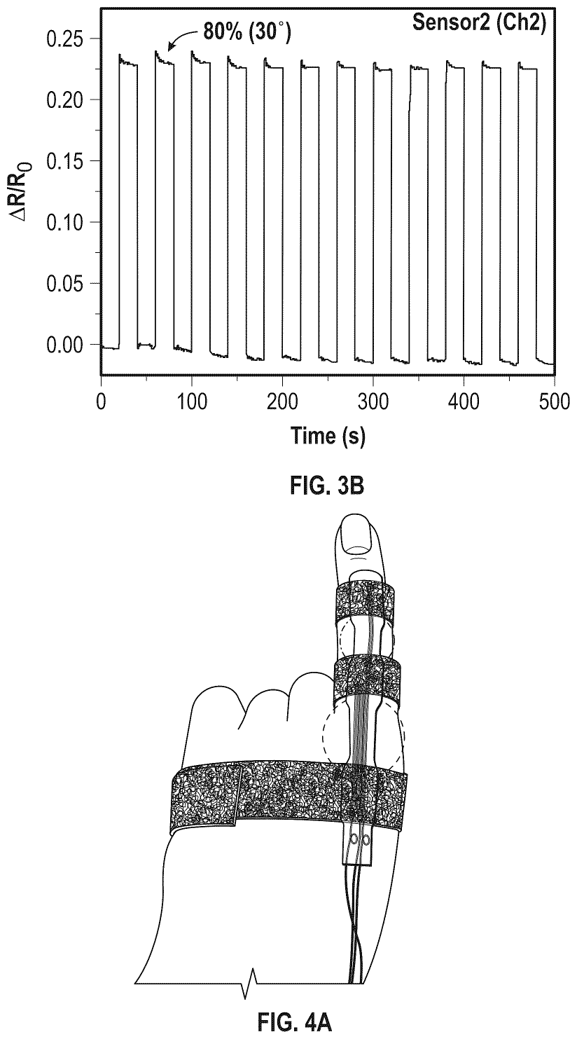

FIGS. 3A-3B show the sensor responses (ΔR/Rₒ) of sensors 1 and 2 as they are subjected to multiple cycles of applied strains, ranging from 0% (i.e., 0°) to about 80% (i.e., 30°), alternatively for twenty seconds (20 s) duration, over twelve cycles. The sensor responses change consistently over the multiple measurement cycles, yielding about 0.135 (sensor 1) and 0.245 (sensor 2). A magnified single response pulse for sensors 1 and 2 between fifteen seconds (15 s) and forty-five seconds (45 s) in FIGS. 3A and 3B, respectively. The insets show the magnified plot of the sensor responses, determining their rise and fall regions (i.e., response and recovery) that indicate the changes happen in four tenths seconds (0.4 s: response time) and two tenths seconds (0.2 s: recovery time). From the single response pulse for the sensor, the signal-to-noise ratio (SNR) can be obtained, eventually determining the measurement resolution (i.e., minimum detectable strain), which is another essential parameter to determine the sensor performance. Generally, the low SNR of a sensor causes a sensor performance limitation, hindering them from measuring small strain changes. The SNR expressed in dB can be calculated using the equation, SNRdB=20 log 10((ΔR/R_{signal})/(ΔR/Rₙₒᵢₛₑ)), where ΔR/R_{signal} is the fractional change in resistance due to applied strains and ΔR/Rₙₒᵢₛₑ is the standard deviation of the resistance fluctuation at those strains. The calculated SNRs for sensors 1 and 2 were 69 and 66.5 dB, respectively, and the measurement resolutions were 0.023 or 2.3% (sensor 1) and 0.0043 or 0.43% (sensor 2), which can be determined as (ΔR/Rₙₒᵢₛₑ)/sensitivity, where the sensitivity was determined from FIG. 2. Furthermore, we tested the sensor durability with a custom-built bending tester. Sensors 1 and 2 were tested together simultaneously between 15° and 30° for one thousand cycles, and showed very reliable results.

The performance of the strain sensor in terms of GF (˜3), SNR (˜69 dB), and measurement resolution (0.43%) is outstanding compared to other stretchable and flexible strain sensors based on EcoFlex, DragonSkin, and PDMS substrates, especially considering the stretchable range and simple fabrication process. Two different types of sensors (i.e., capacitive and resistive) were fabricated using EcoFlex-carbon black (CB) elastomer composites and measured the strain up to 500%. The sensor stretchability without failure operation is very high among EcoFlex-based strain sensors, but it did not translate into correspondingly high GF (capacitive type: 0.98, resistive type: 3.37 at the 500% strain). In the case of EGaIn with EcoFlex substrate, highly stretchable strain sensors (˜550% strain) were demonstrated with GF of 4.95. The sensor includes many microchannels that can suffer from the complicated device fabrication methods due to the lithography process. Also, the sensor had trouble detecting strains in a small area and having multiple sensing channels in a single device because of the relatively big size of the sensor resulting from the numerous microchannels. In the meantime, new methods were reported for the digital printing of multi-layer using conductive and stretchable ink composed of Ag flakes, EGaIn, and styrene-isoprene block copolymers to fabricate highly stretchable strain sensors which can detect up to 600% strain. However, the sensor has a poor GF of 0.9 (˜600% strain), although the printing method is a good approach for complex multi-layer circuit architectures. Hence, considering the overall aspects of the sensor's performances (i.e., GF, SNR, measurement resolution, and response time) and the simple fabrication process, our sensor is outstanding. In addition, architecture for two sensors in a single device allows for fast and accurate detection of two joint movements, resulting in a strong candidate for wearable and robotic motion monitoring systems to measure strain in real-time.

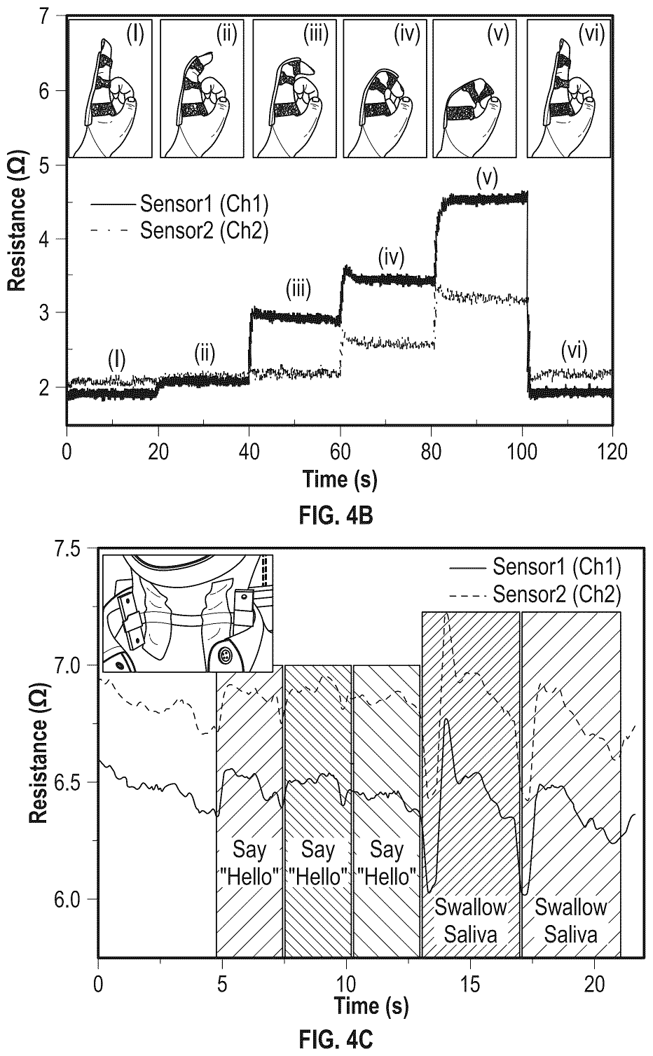

Furthermore, the strain sensor was utilized to monitor the motions of a human finger (PIP and MCP joints) for practical application. The sensor was physically attached to the Velcro strap with silicone epoxy/sealant except for the test areas (PIP and MCP joints) to apply various strains with different finger movements to the sensor, as shown in FIG. 4A. These test areas were able to be bent freely as the joints moved. The various movements of PIP and MCP joints were monitored by sensors 1 and 2, respectively. The sensor was connected to the simple voltage divider circuit to read the finger movements. National Instruments™ (NI) data acquisition (DAQ), including NI SCB-68 and NI USB-6251, supply 5 V DC input voltage to measure the voltage changes of the sensor from the finger movements. The obtained output voltage can be converted to resistance value using equation R=Vₒᵤₜ×R_{ref}/(Vᵢₙ−Vₒᵤₜ). FIG. 4B shows the pictures of the fingers with the attached sensor and the sensor responses as it is subjected to the various finger movements. In this demonstration, there are six sections (i.e., (i)-(vi)) for the test, between zero and one hundred twenty seconds (0 and 120 s), alternatively for twenty seconds (20 s) duration, and the attached sensor 1 and 2 (i.e., Ch1 and Ch2) monitor PIP and MCP joints, respectively. Section (i) is the start point before the finger movements, and section (vi) is the endpoint after the demonstration. Section (ii) to (iii) shows that only the PIP joint moved while the MCP joint maintained its original position, and thus, the only resistance of sensor1 (Ch1) changed in these sections. Section (iv) to (v) shows that both PIP and MCP joints moved together, changing their resistance on sensors 1 (Ch1) and 2 (Ch2), respectively. Hence, the sensors clearly distinguish two different joints of PIP and MCP simultaneously with our novel architecture (i.e., two sensors in a single device). In addition to this work, the sensor was attached to the human vocal cord to monitor its movement as another practical application. The sensor could monitor vocal cord movement while speaking Hello and swallowing saliva, as shown in FIG. 4C. This again highlights an excellent capability for applications in wearable devices and robotic motion monitoring systems, leveraging the superior conductivity of EGaIn and the high deformability of EcoFlex.

### Novel Architecture of Sensor Design

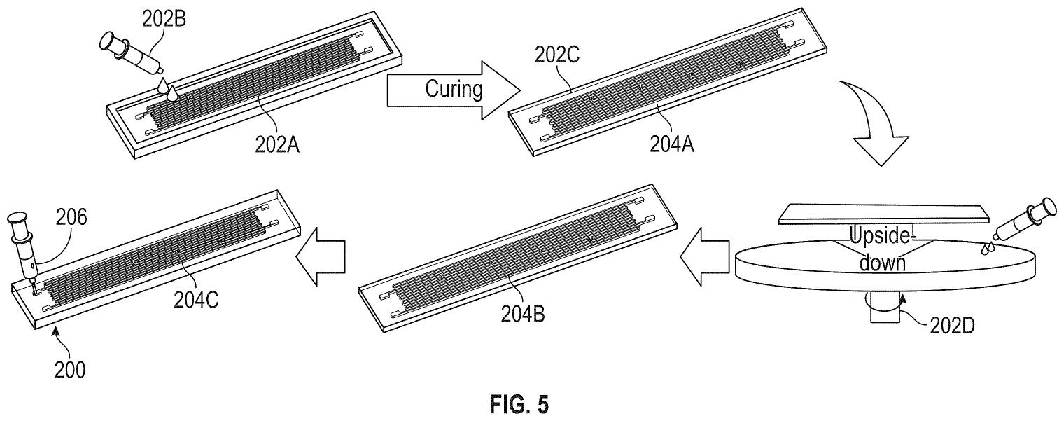

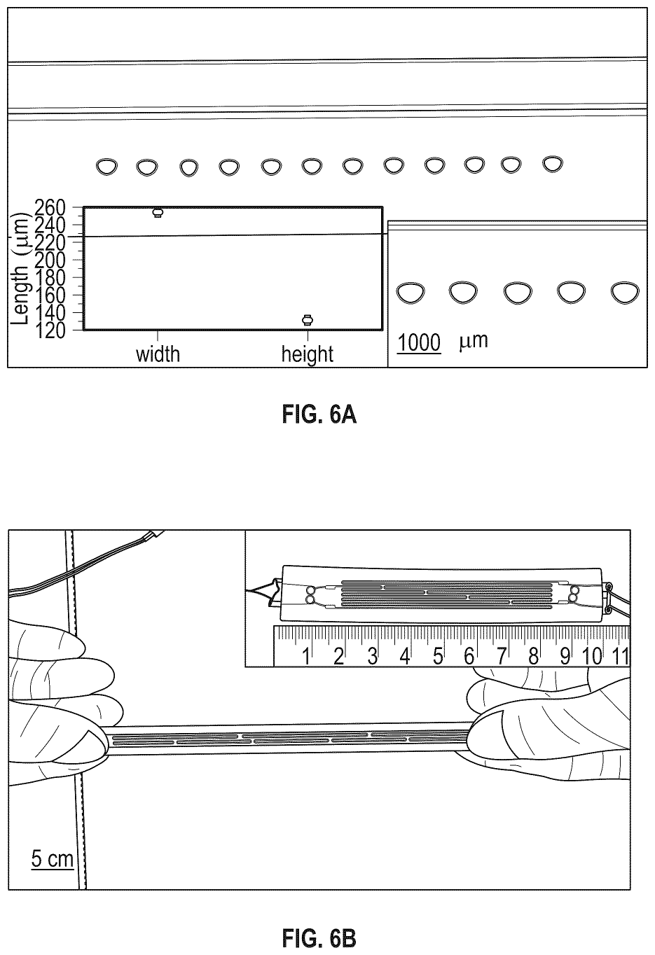

The improved pressure sensor 200, featuring optimized microchannels filled with EGaIn on an EcoFlex substrate 204C, showcases a novel approach to microchannel architecture. The microchannels 204A-204C of the sensor 200 were fabricated based on the reported procedure with the experimental details briefly depicted in FIG. 5. EcoFlex 00-30 prepolymer 202B was poured into the plastic mold 202A and left to cure at room temperature for 4 hours to obtain the open microchannel 204A. The substrate 202C with an open microchannel 204A was attached to the spin-coated silicone backing layer 202D in an upside-down direction to cover the microchannels 204A. The air-filled microchannels 204B were obtained by following the curing of the backing layer 202D. Liquid EGaIn alloy 206 was introduced to the microchannels 204C using a syringe injection method, and the fabrication was completed by connecting copper wires to each sensing channel. All the detailed steps for the fabrication were also described in the Method part. The microchannel dimensions were analyzed, as depicted in FIG. 6A. The average width and height measured 254 and 130 μm, respectively, with less than a 2.5% error compared to the intended dimensions of 250×125 μm. The encapsulation of EGaIn in the microchannel 204C was confirmed by extremely stretching the sensor 200, as shown in FIG. 6B.

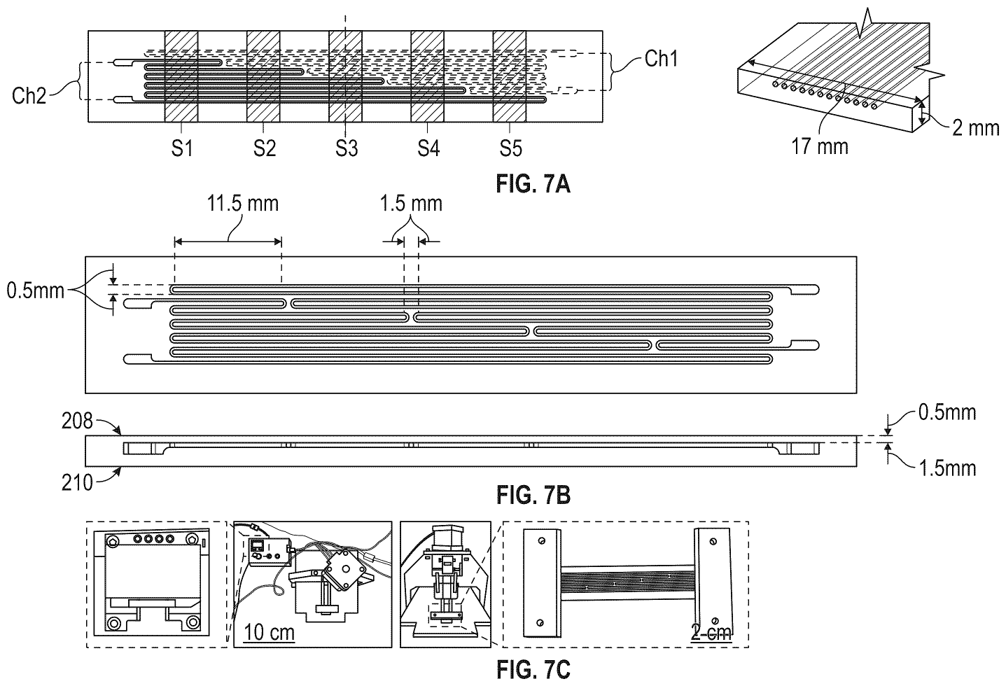

FIGS. 7A-7B illustrate the pressure sensor design, and FIG. 7C shows pictures of the pressure measurement setup and the sensor. The sensor consisted of two sensing channels, individually marked in FIG. 7A. The sensor configuration can be systematically divided into five discrete sections, as depicted in FIG. 7A, based on the novel approach to architecting microchannel. Each section consists of 12 microchannels in total, with the combination of sensing channel 1 (first-type line) and sensing channel 2 (second-type line). The number of microchannels of sensing channel 1 (first-type lines) increases from two to ten with an interval of two, while that of sensing channel 2 (second-type lines) decreases from ten to two. The ratio of microchannels of channel 1 over channel 2 in each section thus increases from left to right, while the ratio of microchannels of channel 2 over channel 1 in each section thus decreases from left to right. This proposed novel design allows the sensor to distinguish five different sections very well, resulting in the detection of pressure location. FIG. 7B shows the detailed dimensional information on the sensor design, including the spacing between the microchannels (500 μm) and the sections (1.5 mm). To test the sensor performance, various pressures were applied to the sensor by a custom-built pressing tester, ranging from 0 to 0.1 MPa, as shown in FIG. 7C.

### Sensor Performance Analysis

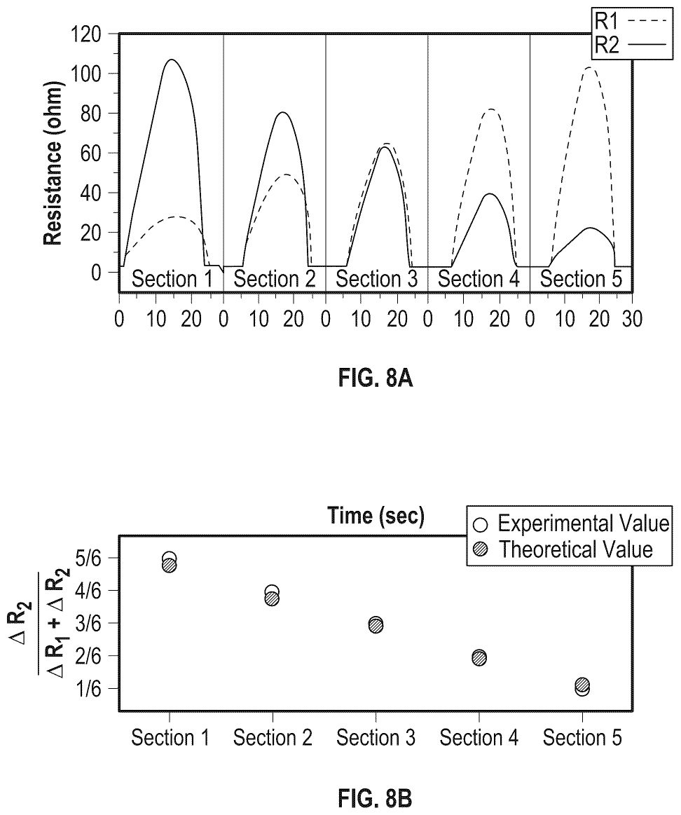

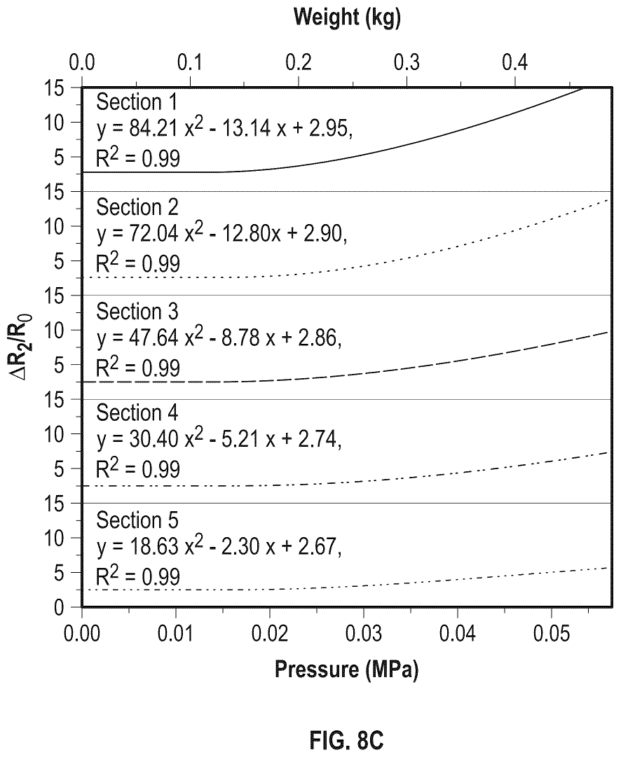

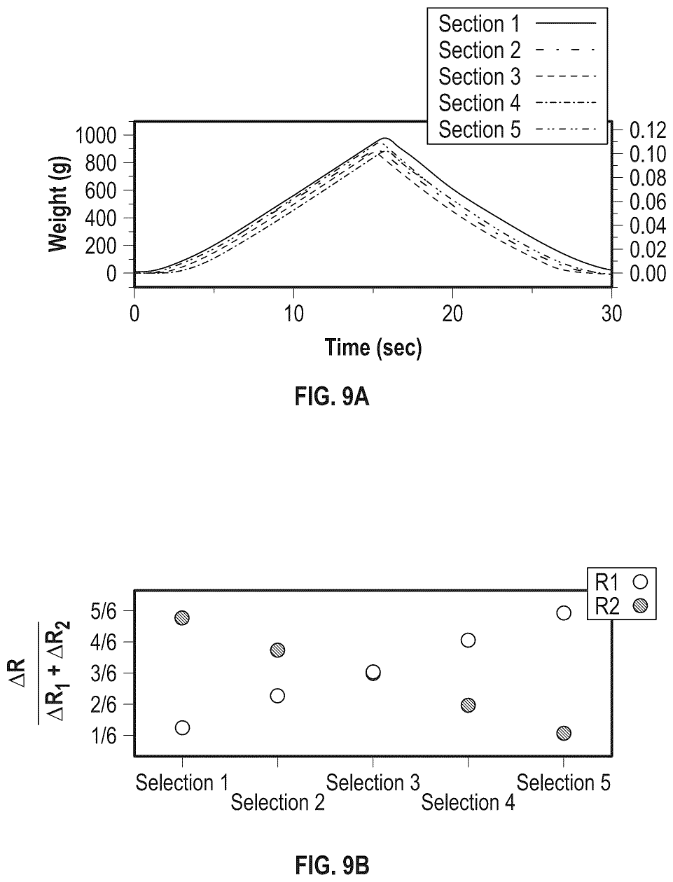

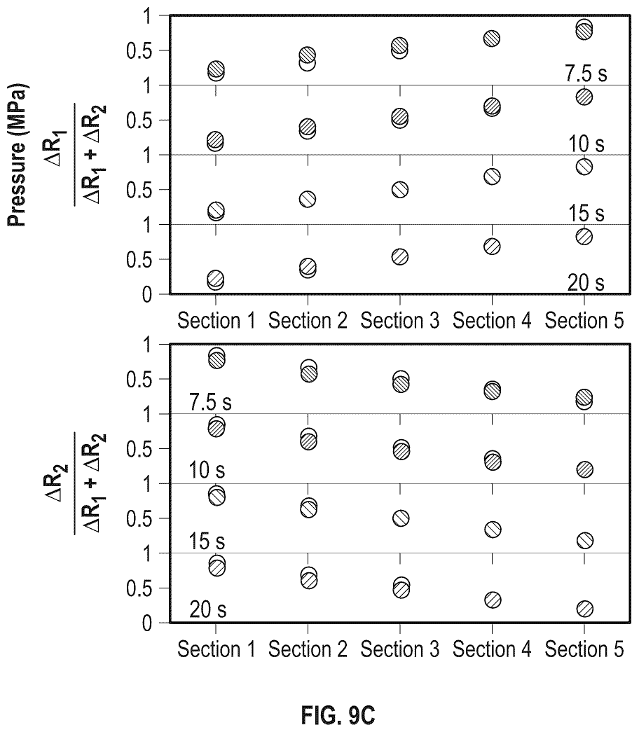

FIGS. 8A-8C show the performance testing results of the pressure sensor. The output resistance R₁ and R₂ represent the response changes of sensing channel 1 and sensing channel 2, respectively, under a gradual loading/unloading pressure test, as shown in FIG. 8A. The detailed information on loading/unloading pressures to each section in the sensor is explained in FIG. 9A. Although the same pressure of up to 0.1 MPa is subjected to each section in the sensor, the responses of sensing channel 1 (R₁, indicated by the first-type line) and sensing channel 2 (R₂, represented by the second-type line) were different across the sections. Notably, in the context of the pressure loading/unloading test, it is evident that the output resistance changes of R₁ exhibited a gradual rise from section 1 to 5, while R₂ registered a decline. This is because of the novel approach to architecting microchannel in pressure sensors, as depicted in FIG. 7A. When pressure is applied to a specific section, it gets uniformly distributed across every microchannel with that section in the sensor, irrespective of their sensing channel affiliations. This uniform distribution induces a consistent resistance change across these microchannels. Thus, the total resistance changes in sensing channels 1 and 2 within the pressed section are the combined changes from each microchannel. Based on this, the pressure location index (ΔR/(ΔR₁+ΔR₂)) can be calculated by considering the ratio of the resistance changes in sensing channels 1 and 2 (ΔR₁ or ΔR₂) in relation to the combined resistance change in both sensing channels (ΔR₁+ΔR₂). In FIG. 8B, the experimentally determined pressure location index (shown in third-type dot) for different sections of sensing channel 2 exhibited a linear decrease, closely matching the theoretically derived value (depicted in first-type dot). The theoretical value was calculated based on the expected resistance change ratios for the sensing channels. FIG. 9B further illustrated that the pressure location index between sensing channel 1 (first-type dot) and sensing channel 2 (second-type dot) displayed a contrasting trend, confirming the effectiveness of the sensor's design. Moreover, irrespective of the specific timing of the measurements (or the exact pressure at that moment), the pressure location indexes for sensing channel 1 (FIG. 9C, upper graph) and sensing channel 2 (FIG. 9C, lower graph) consistently aligned with the theoretical expectations. This highlights the reliability of the pressure location index as a dependable indicator of pressure location.

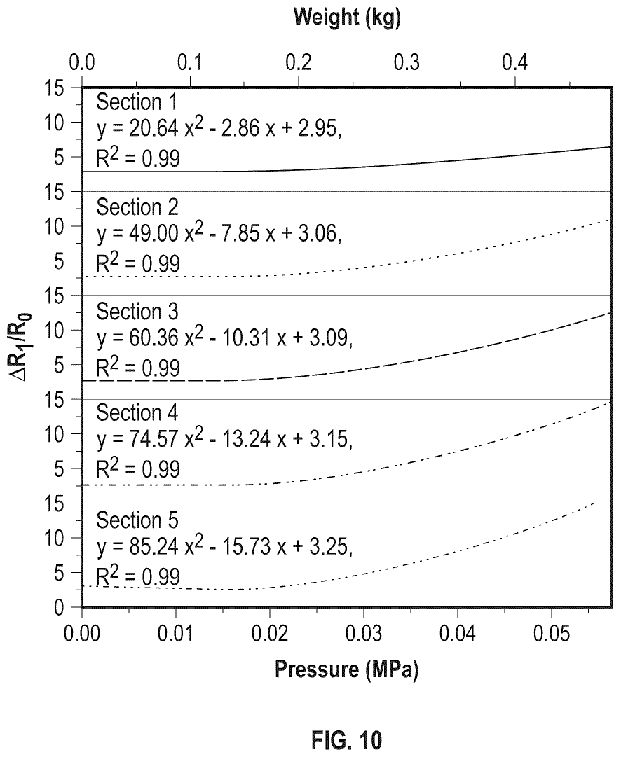

The performance of the sensor was further assessed, as depicted in FIG. 8C, where the pressure was gradually applied up to 0.05 MPa. On the y axis, R₀ represents the initial resistance value, and ΔR₂ denotes the resistance change observed in sensing channel 2 (Sensing channel 1's response is showcased in FIG. 10). The exerted pressure was derived from the weight loaded using the equation P=F/A. Here, P stands for pressure, and F for force. The contact area, A, was measured to be 85 mm² (17×5 mm²). The relative sensor response ΔR₂/R₀ (or ΔR₁/R₀ in FIG. 10) was plotted against the applied pressure (or the applied weight) and fitted with a parabolic equation y=Ax²+Bx+C, where y is the relative sensor response, and x is the applied pressure. In this context, a sensor's sensitivity can be derived from the slope of the output (ΔR/R₀) concerning the applied pressure ΔP, represented as Sensitivity S=(ΔR/R₀)/ΔP. This sensitivity parameter, an essential aspect of sensor performance, was calculated to be 66.07 MPa⁻¹ under an applied pressure of 0.05 MPa. The outstanding sensitivity of the sensor is attributed to the low Young's modulus (˜0.5 MPa) of the EcoFlex 00-30. The pressure given to the soft EcoFlex matrix induces the volume changes in the microchannels, resulting in a change in the resistance of the liquid metal. Even subtle pressure can directly influence the resistance variation of the liquid metal, thus achieving high sensitivity.

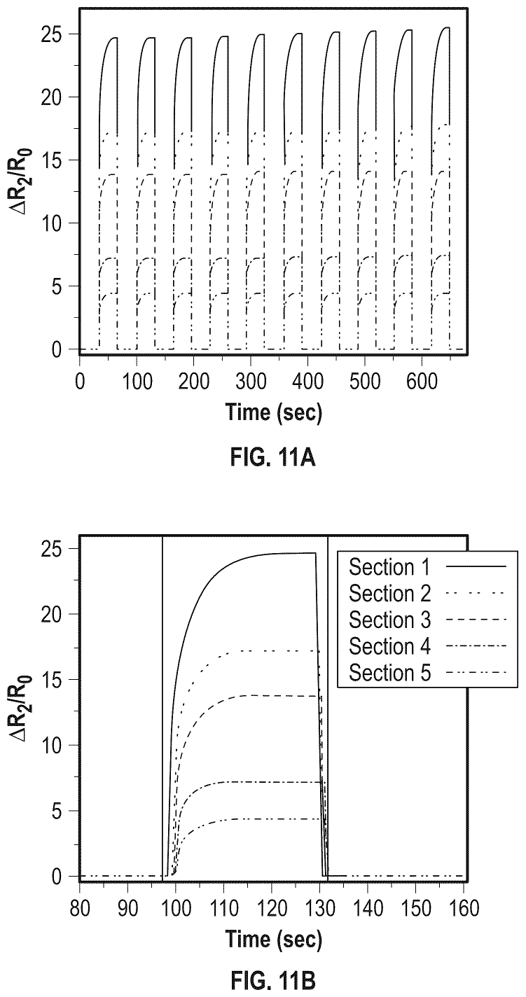

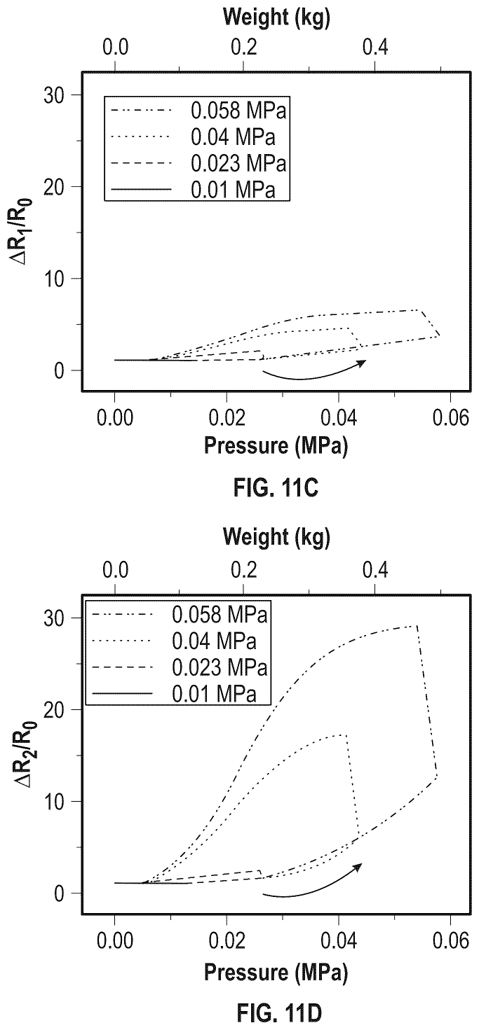

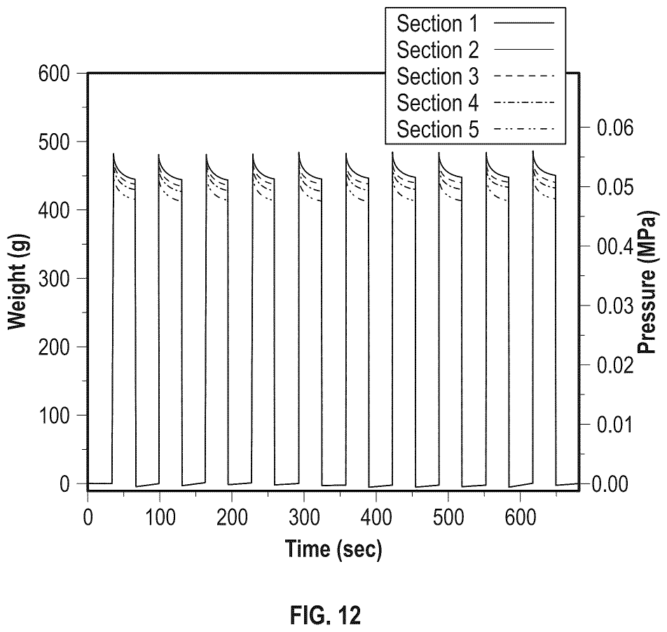

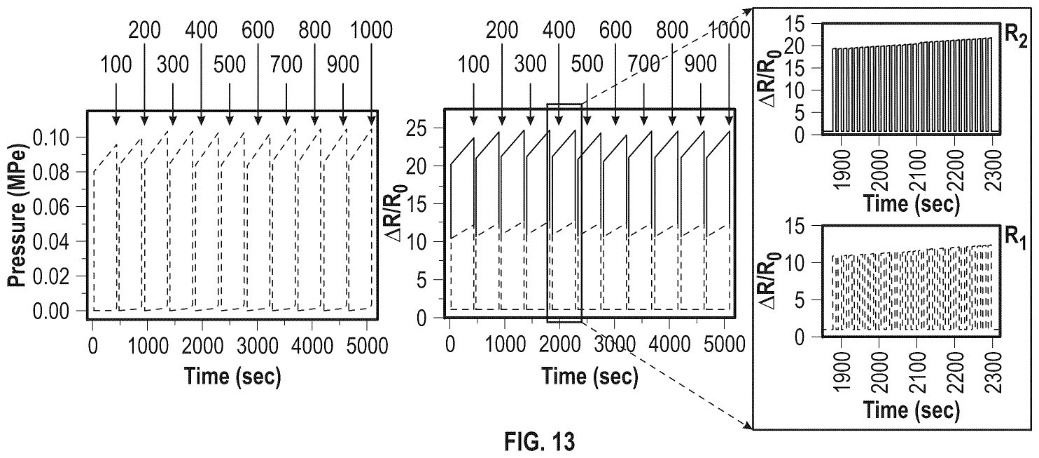

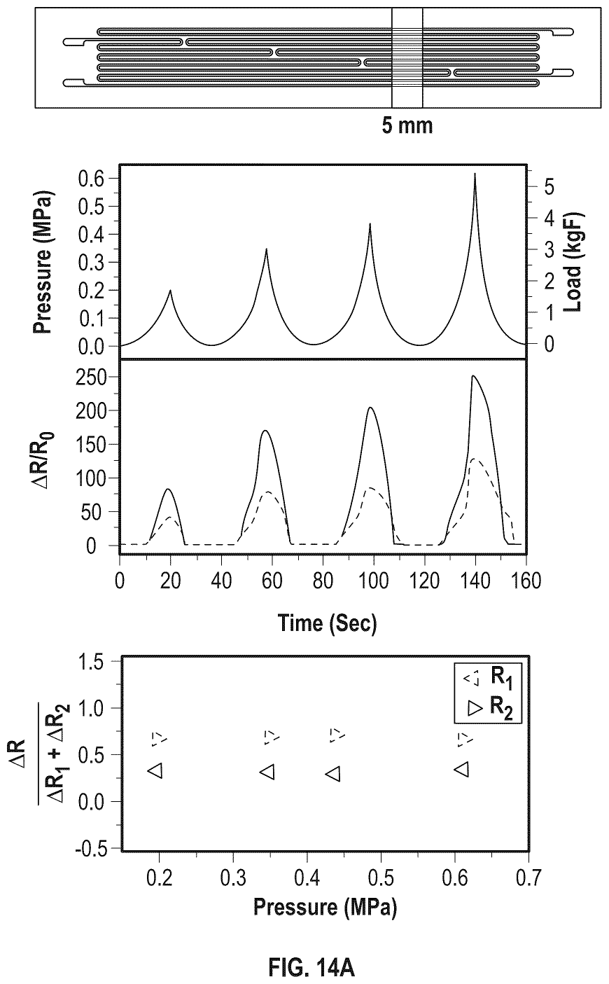

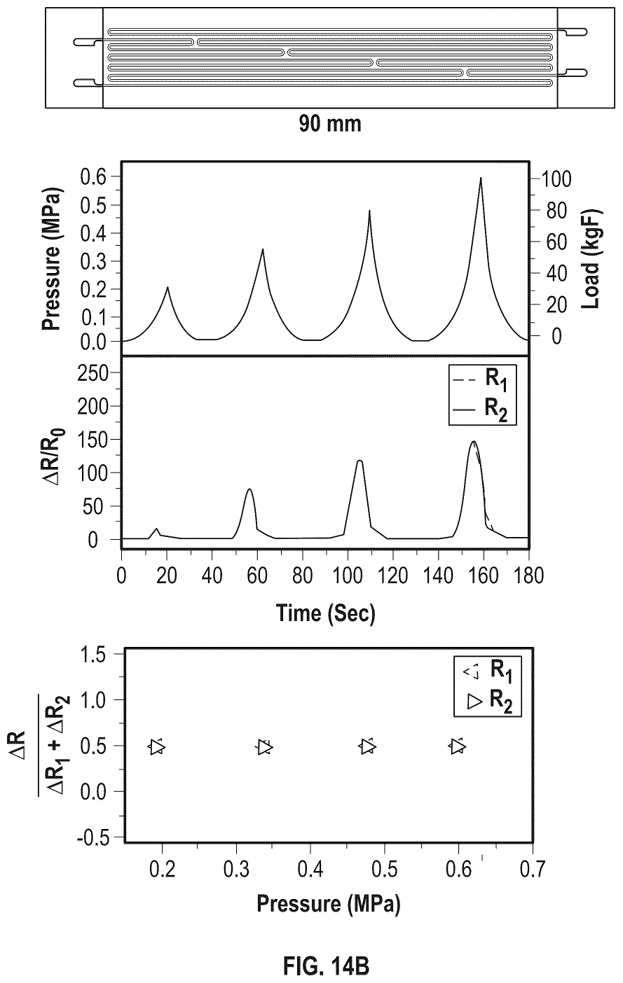

A cyclic test of the sensor was conducted, and the results are displayed in FIGS. 11A-11B. FIG. 11A shows the sensor's responses across sections 1 to 5 during ten cycles of applied pressure ranging from 0 to 0.05 MPa. The progression of the applied pressure (i.e., weight) during the test is detailed in FIG. 12. The results demonstrate that the sensor consistently performs across multiple measurement cycles in all sections 1 to 5 under the applied pressure, as shown in FIG. 11A. FIG. 11B provides a zoomed-in view of the single pulses, specifically capturing the time frame between 80 and 160 seconds from FIG. 11A. From the data obtained from the single pulses shown in the results, the signal-to-noise ratio (SNR) can be calculated. The SNR is crucial in determining the measurement resolution, which indicates the smallest change the sensor can detect. The measurement resolution is one of the critical parameters when evaluating the overall sensor performance. A lower SNR can limit the sensor's capacity to discern minor changes in pressure, underscoring the significance of this ratio in sensor performance. The SNR (dB) is determined using the formula SNR (dB)=20 log₁₀((ΔR/R_{signal})/(ΔR/Rₙₒᵢₛₑ)). Here, ΔR/R_{signal} represents the fractional resistance changes due to applied pressures, while ΔR/Rₙₒᵢₛₑ indicates the standard deviation of the resistance fluctuation under those pressures. See Kim et al., “Piezoresistive graphene/P (VDF-TrFE) heterostructure based highly sensitive and flexible pressure sensor.” ACS Appl Mater Interfaces 2019, 17, 16006, which is hereby incorporated by reference in its entirety herein. The sensor exhibited an average SNR value of 72.10 dB and a measurement resolution (ΔR/Rₙₒᵢₛₑ/sensitivity) of 0.056 kPa, highlighting its superior performance. The durability of the sensor was evaluated by conducting 1000 cyclic tests in section 4 with a pressure of 0.08 MPa, and the results are depicted in FIG. 13. Even under harsh conditions, the tested result was very reliable. A cyclic test with varying applied pressures from 0.01 to 0.06 MPa was conducted to check the sensor's reliability, and the response of sensing channels 1 and 2 in section 5 pressed with varying pressures is depicted in FIGS. 11C-11D. Remarkably, the resistance-changing curves for both R₁ and R₂ overlapped consistently across different applied pressures. This result indicates that the fitted parabolic equations depicted in FIG. 12C accurately represent a sensor performance with high reliability under wide pressure ranges. The sensor was further assessed by pressing with a larger dimension and a higher pressure of up to 0.6 MPa, and the following results are displayed in FIGS. 14A-14B. FIG. 14A depicts resistance variation under pressure localized to section 4 (17×5 mm²), while FIG. 14B illustrates the resistance variation resulting from pressure applied across all the sections (17×90 mm²) (pressed areas are highlighted with toward the top of the figure). Irrespective of the pressing dimension or the pressure amount, the sensor resistance variations exhibited reliable results, and the related pressure location indexes highly matched the theoretical values, demonstrating the potential of the sensor as a gait monitoring sensor 300.

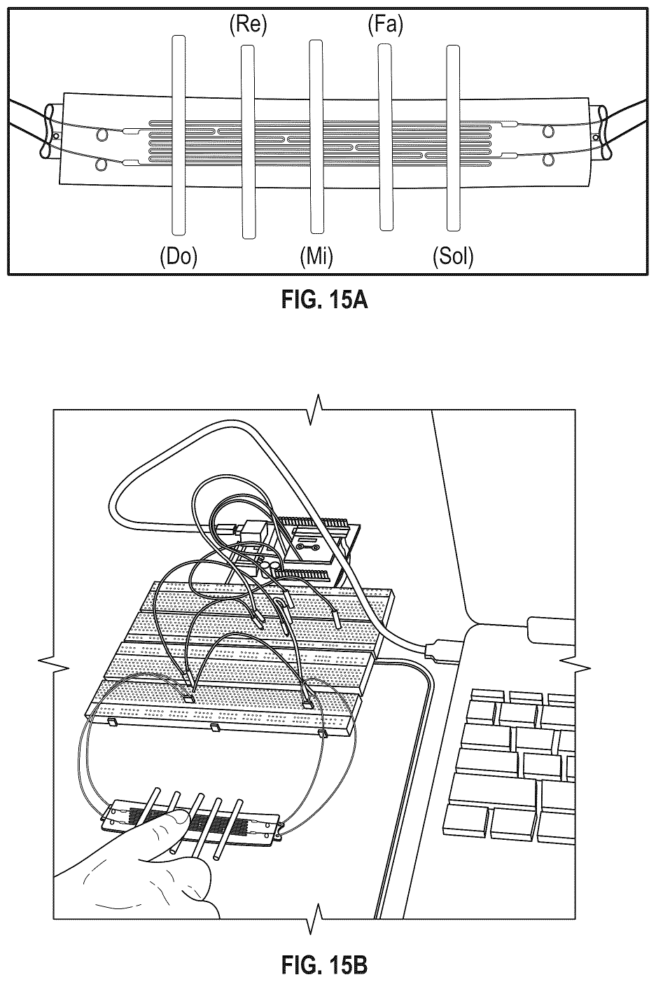

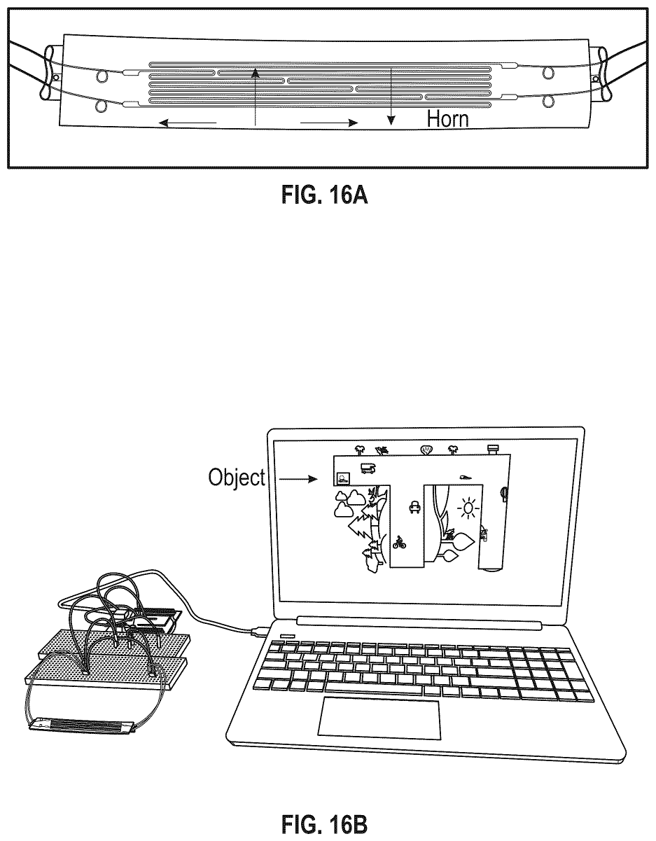

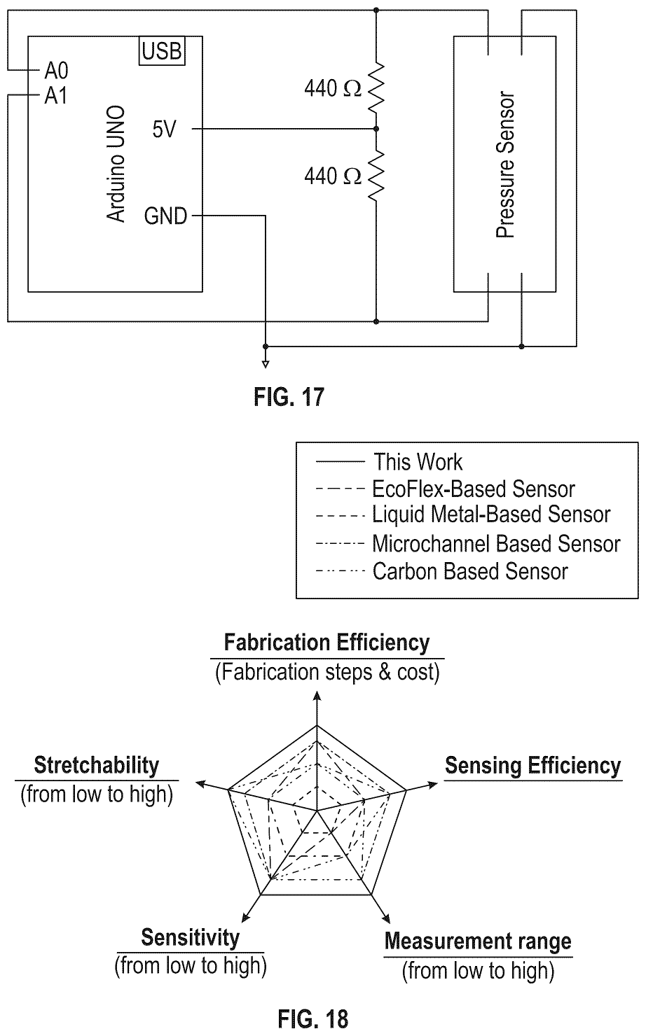

Sensors' extensive sensitivity and ability to accurately discriminate pressure applied location were evidenced by employing two applications: piano keys (FIG. 15A) for playing a piano (FIG. 15B) and a joystick (FIG. 16A) for car control (FIG. 16B). For the experiment, the sensor was connected to Arduino UNO with an electronic circuit, as depicted in FIG. 17, and the sensor sections were discriminated by adjusting the threshold of resistance changes. In both applications, the movies clearly illustrate that the novel microchannel design successfully achieved sectional discrimination, while the remarkable sensor sensitivity allowed it to respond to light finger pressing.

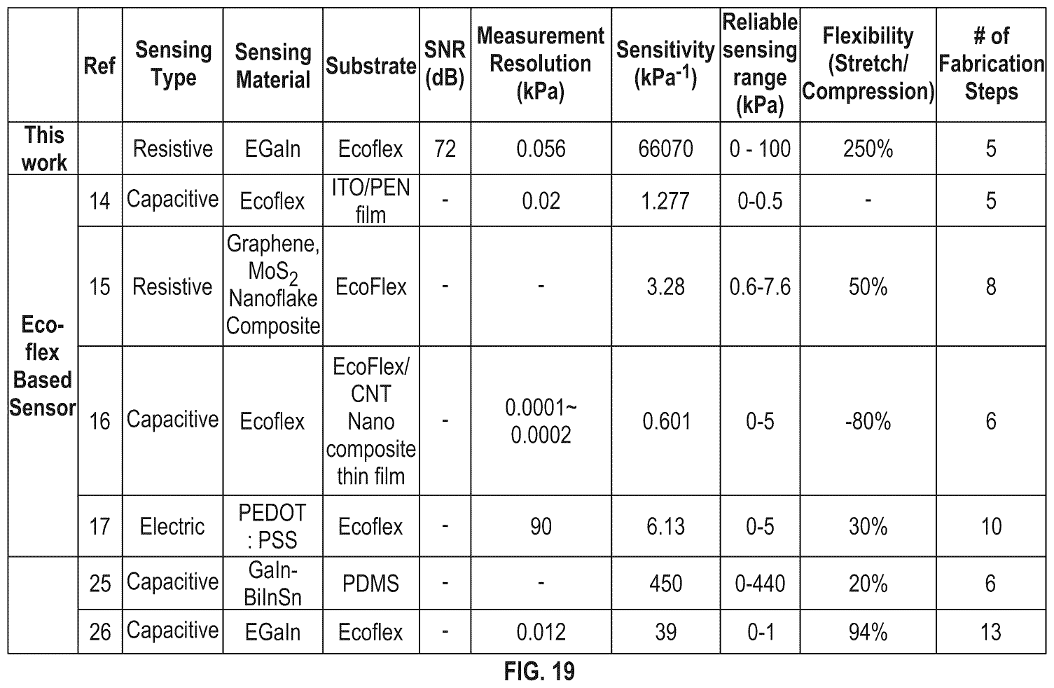

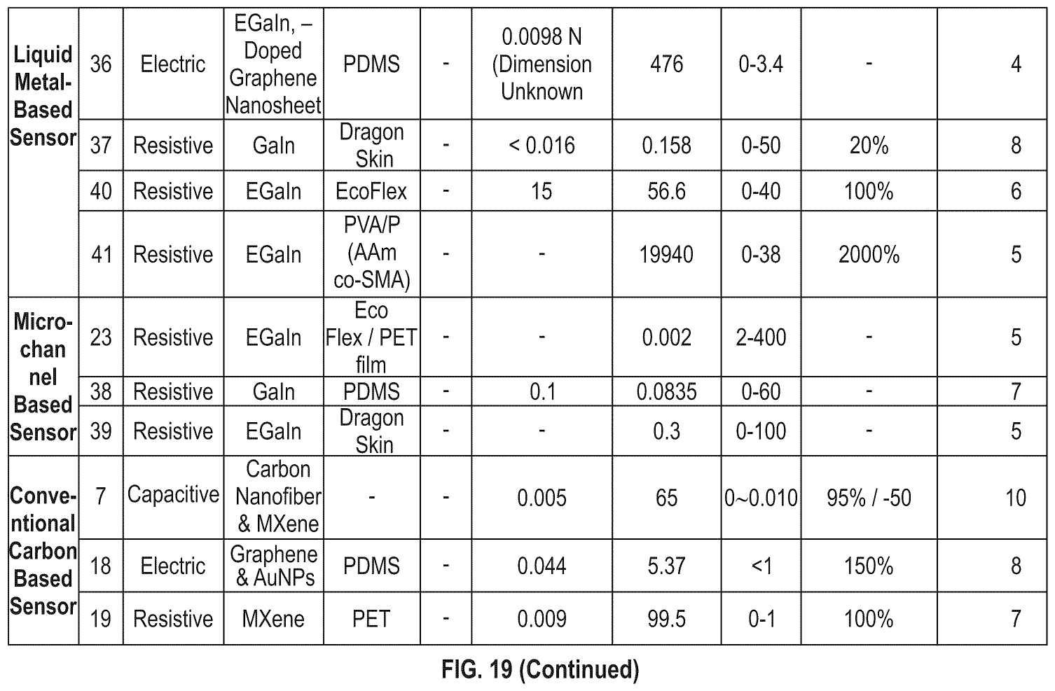

A systematic evaluation of the fabricated pressure sensor's performance and efficiency was conducted, with results compared against previously reported research findings summarized in FIG. 19. The sensor notably distinguishes itself with its extensive and reliable sensing range of 0 to 100 kPa and exceptional sensitivity of 66.07 MPa⁻¹. This remarkable achievement is exemplified in FIG. 8C, where the variations in sensor output resistance across different sections were well-modeled by a parabolic equation, which is an uncommon result compared to previously reported pressure sensors. Many sensors in previous studies exhibited a linear relationship between sensor output and applied pressure within a limited pressure range; thus, this limitation restricted their applicability to a narrow pressure range despite their high sensitivity. In contrast, the sensor, owing to the hyperelastic properties of the EcoFlex substrate, can directly translate even subtle deformations of the substrate into variations in the shape of the EGaIn within the microchannel. This property accounts for the pronounced changes in output resistance across a wide pressure range, thus contributing to the sensor's outstanding sensitivity, considering the materials, fabrication techniques, or sensing methods employed in other sensors.

For a comprehensive comparison, performances of previous works using a pentagonal diagram (FIG. 18) are categorized into four groups: EcoFlex-based, liquid metal-based, microchannel-based, and carbon-based pressure sensors. Compared to previous works, the sensor completely filled the pentagonal diagram, illustrating both its exceptional performance and the cost-effective fabrication method. In contrast to other microchannel-based sensors that often rely on complex soft lithography for fabrication, the method successfully encapsulated the liquid metal within microchannels using a 3D-printed plastic mold, offering a simpler and more economical approach. The durability of the EcoFlex substrate allowed the device to stretch up to 250% without any EGaIn leakage, resulting in remarkable stretchability, sensitivity, and a wide reliable sensing range. Furthermore, the innovative microchannel architecture combined with pressure-sensitive materials elevated the sensor's capabilities, enabling it to distinguish the pressure-applied location precisely with a single device, and widen the applicable area.

### Application of the Pressure Sensor: Wearable Gait Monitoring Sensor

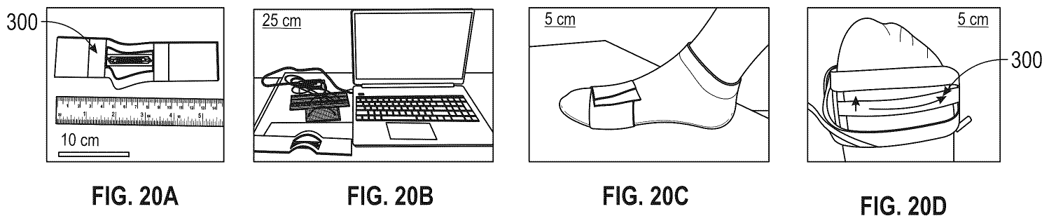

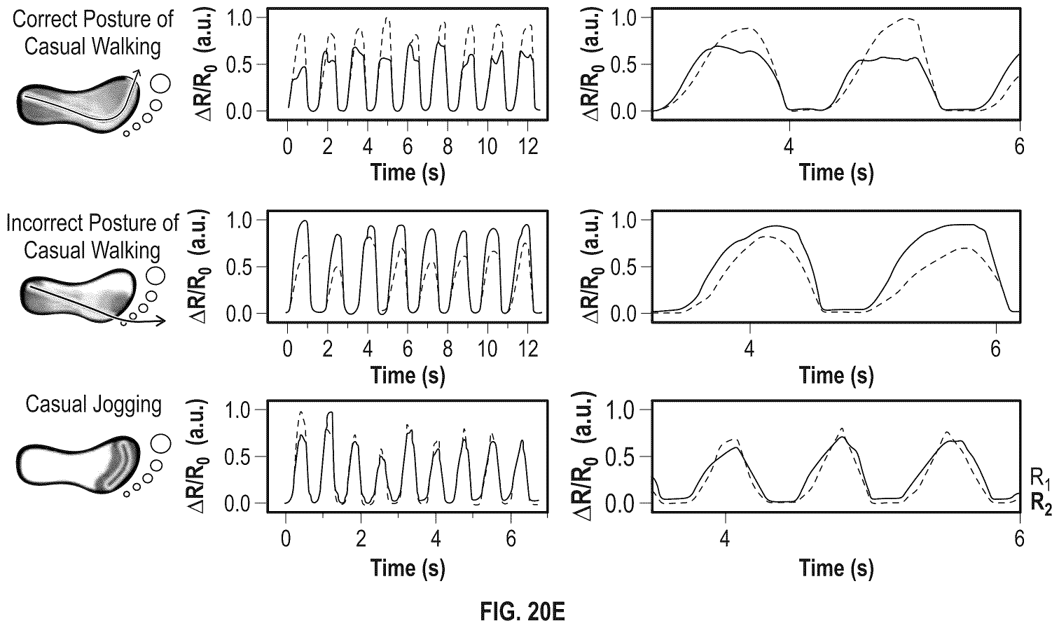

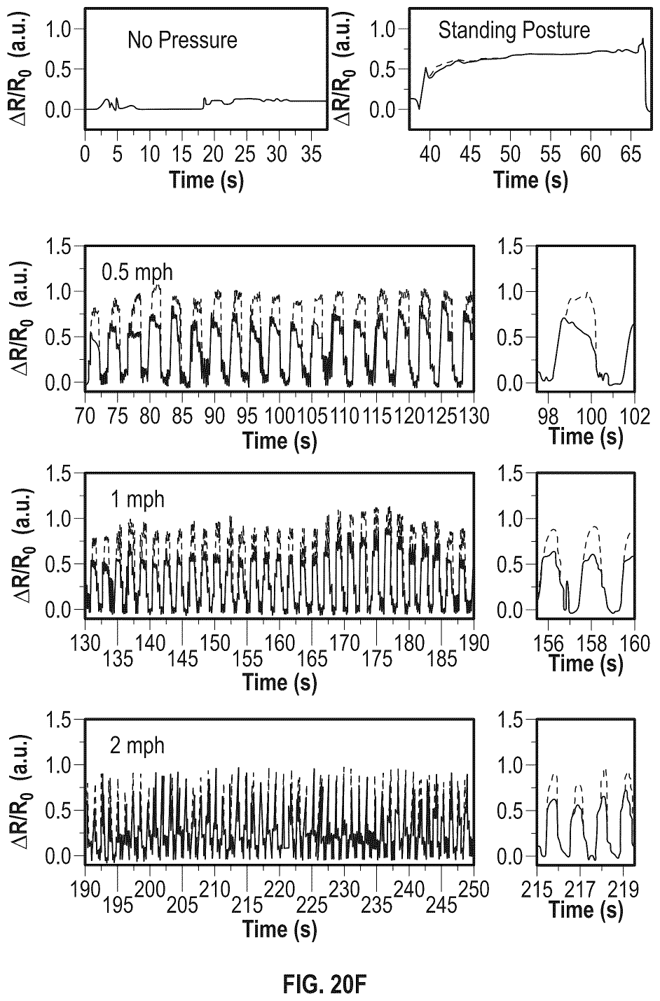

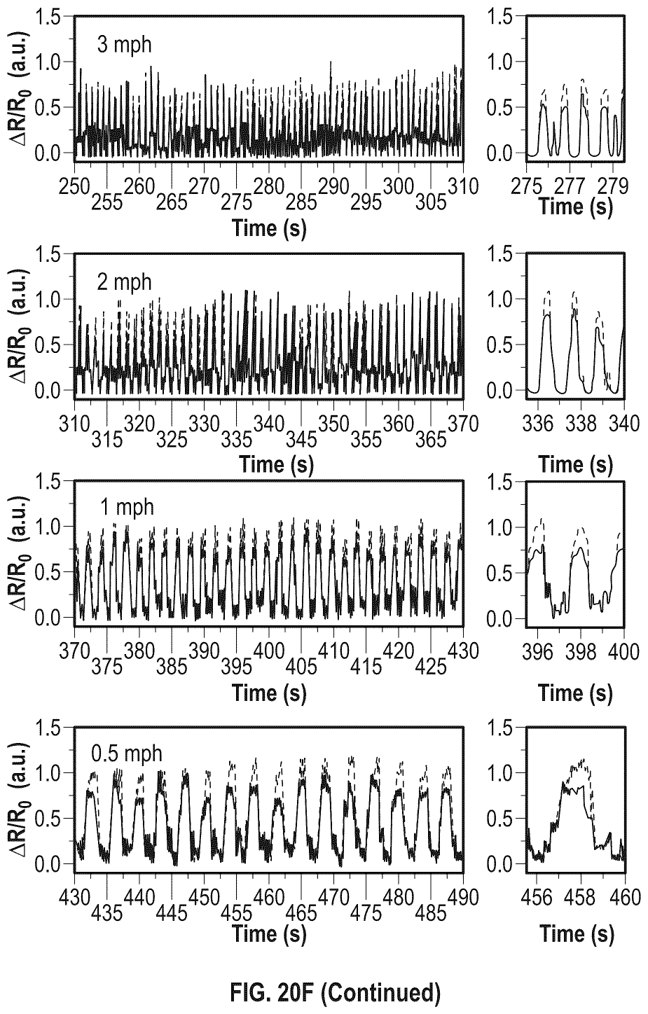

The pressure sensor has a practical application in gait monitoring, with a specific focus on analyzing the dynamic gait patterns of a single leg during motion. The sensor was affixed to the subject's right foot using a Velcro strap, as depicted in FIG. 20A. A detailed configuration illustrating the connection with an Arduino is provided in FIG. 20B. The sensor, attached via the Velcro strap, was strategically placed on the subject's right foot (underneath the ball joint of the forefoot, along distal ends of metatarsals) to span from beneath the thumb (section 1) to beneath the little toe (section 5), as illustrated in FIGS. 20C-20D. Pressure distribution was evaluated on the subject's right foot during both stationary and dynamic phases by recording the resistance changes of R₁ and R₂ throughout the gait cycle, with Rₒ denoting the initial values of R₁ and R₂. The sensor placed on the forefoot facilitated an in-depth examination of the pressure distribution across the walking cycle. The subject was instructed to perform different gait postures, including correct walking (1 mph speed), incorrect walking (1 mph speed), and casual jogging (5 mph speed). The resistance changes corresponding to each posture are depicted in FIG. 20E. The results revealed distinct and specific patterns associated with different gait postures, and these patterns remained consistent throughout each testing duration, highlighting the sensor's capability to distinguish gait postures effectively.

To elucidate the detailed comparison between the postures, the graphs of resistance changes from 3.5 to 6 sec were magnified as highlighted within yellow boxes. In the correct walking cycle, R₁ (first-type line) initially registered higher values than R₂ (second-type line), which can be interpreted as more pressure being applied to section 5, i.e., to the little toe. After a few seconds, the resistance values of R₁ and R₂ underwent a reversal, indicating the pressure was shifted from the little toe (section 5) to the thumb (section 1), as denoted by the arrow in the foot diagram on the left side of the graph. On the other hand, in incorrect walking cycles, R₁ consistently exceeded R₂, suggesting sustained pressure on the little toe throughout the cycle. Meanwhile, in jogging cycles, R₁ and R₂ exhibited similar values, signifying uniform pressure distribution across the forefoot area.

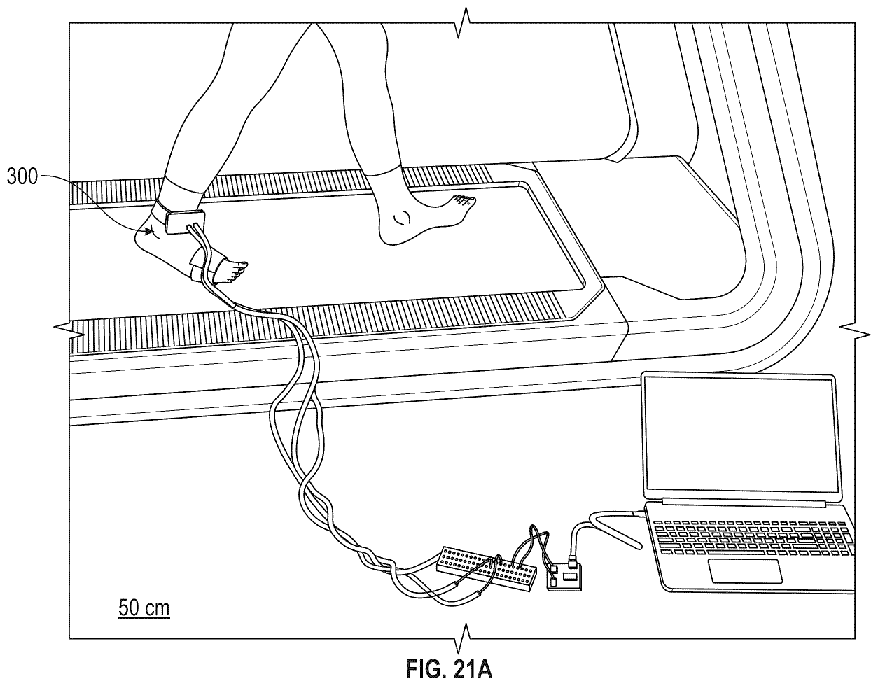

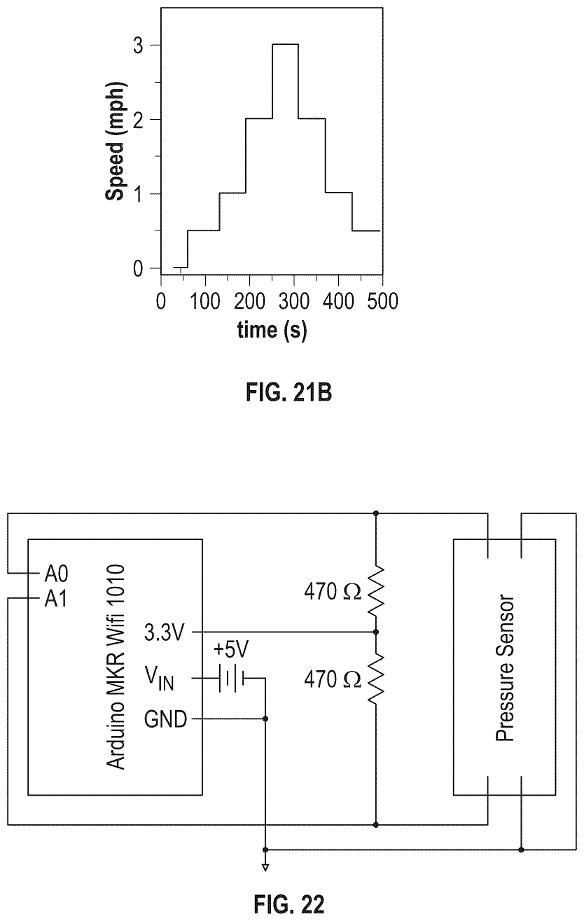

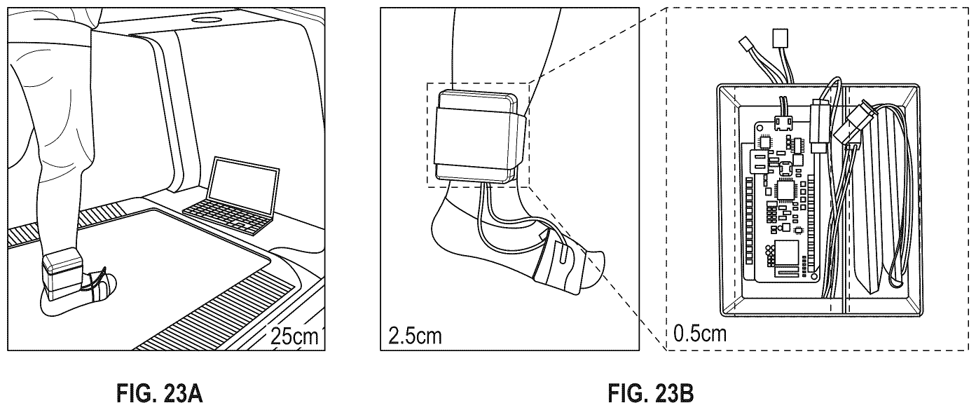

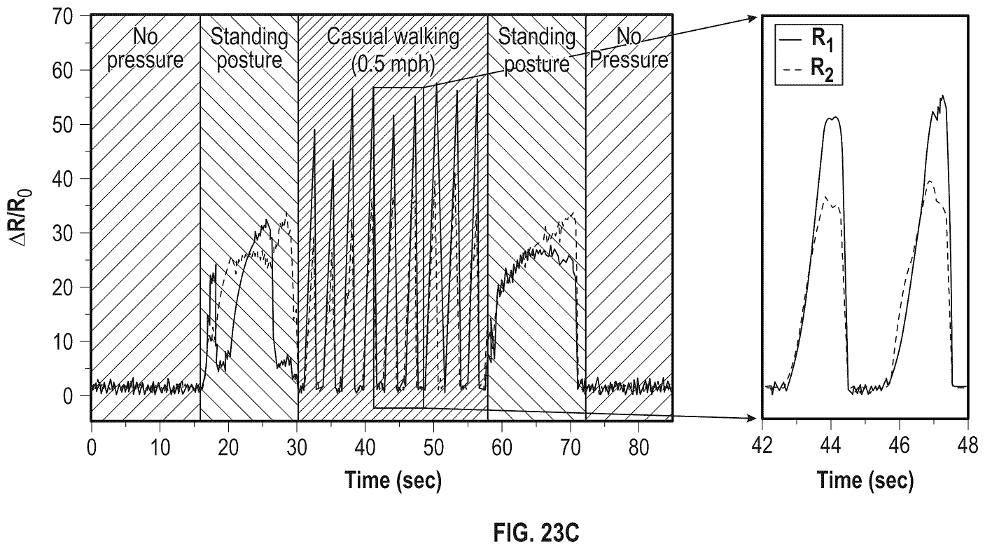

To further explore the sensor's capabilities, the subject was tasked with walking on a treadmill at varying speeds (ranging from 0 to 3 mph) while maintaining the correct walking gait. Detailed images of the experimental setup and speed variations can be found in FIG. 21A-21C. The resistance changes observed during this test are graphically depicted in FIG. 20F. When the sensor was not pressed (0˜37.5 s) or evenly pressed (standing posture, 37.5˜67.5 s), R₁ and R₂ showed comparable values. While walking at different speeds, the sensor consistently exhibited a specific pattern reflective of the correct walking posture. Regardless of the walking speed, R₁ (first-type line) initially showed prominence, which was then reversed by R₂, as clearly evident from the highlighted graphs. Under the casual walking condition (1 mph), real-time wearable gait monitoring results were recorded on the laptop (see FIGS. 21A-21B), and the monitoring system distinguished the correct posture of casual walking very clearly. The sensor's utility was enhanced by developing a wireless data acquisition system using an Arduino MKR WiFi 1010 microcontroller with an electronic circuit, as depicted in FIG. 22. The sensor was fixed to the subject's foot while the microcontroller was attached to the ankle (FIGS. 23A-23D). The microcontroller transferred the collected data with various postures to a personal computer via an IP address and WiFi connection. As can be seen in FIG. 21A, the sensor with the wireless system successfully collected real-time data while walking at different speeds. In FIG. 23D, especially in the zoomed-in graph on the right side, the sensor showed a unique pattern of correct walking posture, coinciding with the wired system-based data in FIGS. 20A-20F. This development improved the practicality of using the sensor during walking, thereby underscoring its potential for monitoring individuals with mobility impairments, including those with conditions such as Parkinson's disease or other walking challenges, in their rehabilitation efforts.

From the foregoing, it can be seen that the present disclosure demonstrates, at least, how to make and use highly sensitive pressure sensor tailored for movement (e.g., gait) monitoring applications. The sensor was fabricated using a cost-effective manufacturing process that capitalizes on the impressive ultra-stretchable properties of EcoFlex and EGaIn liquid alloy. The sensor exhibited remarkable attributes, including non-linear changes in resistance when subjected to pressures up to 100 kPa, accompanied by an exceptional Signal-to-Noise ratio (SNR) value of 72 dB. The incorporation of two sensing channels and its innovative design allowed for the simultaneous measurement of both absolute pressure values and the precise localization of applied pressure. Consequently, the sensor displayed outstanding performance in accurately discerning various human gait postures. The outcomes of the sensor testing offer valuable insights, suggesting promising opportunities for its utilization in clinical applications and rehabilitation studies.

From the foregoing, it can be seen that the present disclosure accomplishes at least all of the stated objectives.

### Methods & Examples

To validate the technology of the present disclosure, various experiments were conducted encompassing different postures (standing, overpronation, underpronation, etc). The results demonstrated that the sensor could accurately distinguish between each posture by comparing the output signals from two distinct sensing channels in a single device.

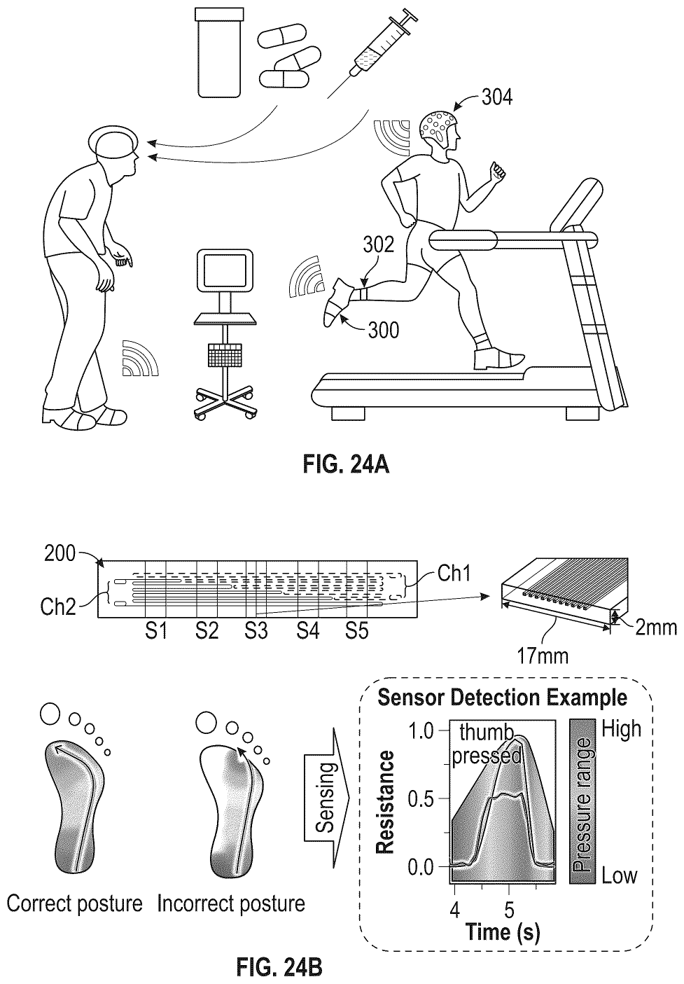

Moreover, during walking trials shown in FIGS. 24A-24B, the gait-monitoring sensor 300 effectively tracked continuous changes in pressure distribution over time, accurately capturing the subject's center of pressure movement. Additionally, it was confirmed that the sensor operated properly even under a weight of 100 kg, attributed to the inherent properties of the materials used, the elastomers and the liquid state conductor.

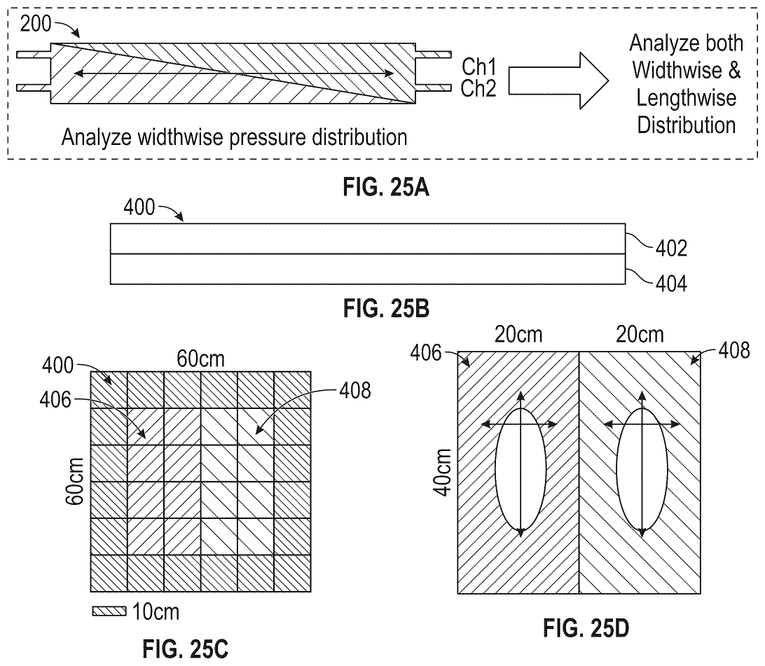

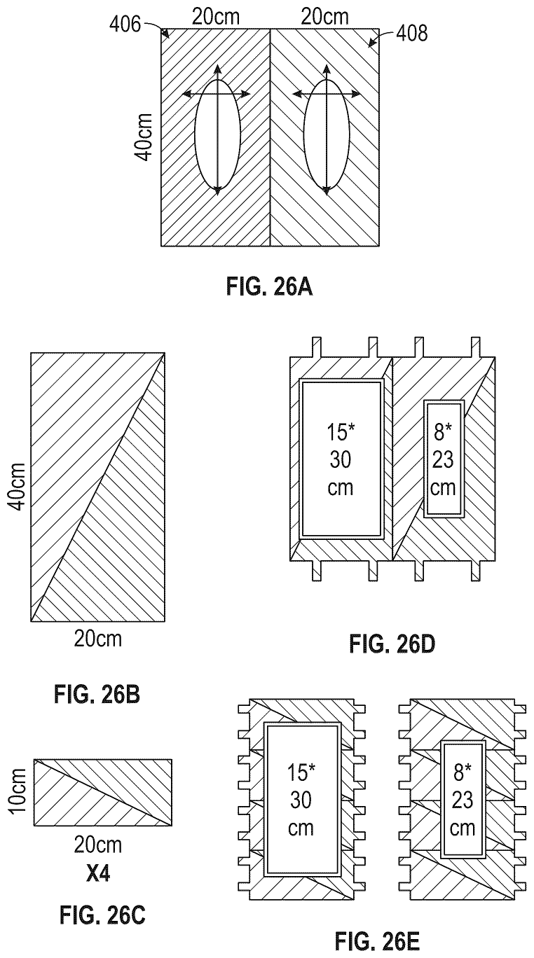

Developing the mat-type pressure sensor 400 according to the dimensions shown in FIGS. 25A-25D and 26A-26E, having 1^{st} and 2^{nd} layers 402, 404, having an expanded number of sensing channels, and having dedicated areas 406, 408 for sensing the correct and/or incorrect posture of left and right feet, showed significant promise for accurately distinguishing pressure distribution across a large dimension, enabling data collection from multiple subjects at once. These experimental findings strongly support the superior performance of the technology compared to existing solutions. Consequently, using a simple sensor system to monitor the gait of high-weight subject in real-time surpasses the results achieved in traditional laboratory settings.

### Pressure Sensor Fabrication

The mold for microchannels was prepared using rigid/durable Vero family photopolymer inks using a 3D printer (Objet500 Connex2 3D printer, Stratasys, Ltd). The dimension of the microchannels was designed as 250 μm×125 μm, with a distance of 500 μm between the microchannels. To fabricate the substrate, EcoFlex 00-30 silicon prepolymer (Smooth-on, Inc) was prepared by mixing the silicon parts with a ratio of 1:1. The prepolymer solution was spin-coated and cured to obtain the silicon-based microchannel substrate. EGaIn liquid alloy (Sigma-Aldrich, Inc) was injected into the microchannels to complete the fabrication process. While filling in the EGaIn with a syringe, an additional syringe needle was inserted into the end of the microchannels to release any trapped air. Once the EGaIn alloy was evenly and thoroughly filled into the microchannels, the fabrication process was completed by connecting 32 AWG-enameled copper wires to the terminal ports of each sensing channel.

### Measurements and Characterizations

The performance of the pressure sensor was measured using a motorized pressure-applying system (shown in FIG. 7C). The motorized system controls the direction, pressed depth (distance), speed, cycles, and duration of the loading/unloading procedure. The sensor data was acquired using an NI USB-6251 data acquisition system along with an NI SCB068 module. The 5 V DC input voltage to the sensor is provided by the NI SCB-68 module, which measures the voltage change caused by the geometric deformation of the EGaIn microchannels induced by pressing the sensor.

### LIST OF REFERENCE CHARACTERS

The following table of reference characters and descriptors are not exhaustive, nor limiting, and include reasonable equivalents. If possible, elements identified by a reference character below and/or those elements which are near ubiquitous within the art can replace or supplement any element identified by another reference character.

TABLE 1
List of Reference Characters

| | |
|---|---|
| 100 | elastomeric pressure sensor |
| 102 | silicone matrix |
| 104 | microchannel |
| 106 | first sensor (e.g., sensor for channel 1) |
| 108 | second sensor (e.g. sensor for channel 2) |
| 112 | first joint |
| 114 | second joint |
| 116 | strain applied region |
| 200 | improved elastomeric pressure sensor |
| 202A | 3D printed mold |
| 202B | silicone mixture |
| 202C | silicone elastomer |
| 202D | spin coated backing silicon layer |
| 204A | open microchannel |
| 204B | air-filled microchannel |
| 204C | liquid EGaIn alloy-filled microchannel |
| 206 | liquid EGaIn alloy |
| 208 | top surface |
| 210 | bottom surface |
| 300 | gait monitoring sensor |
| 302 | transmitter |
| 304 | EEG sensor |
| 400 | mat with improved elastomeric pressure sensor |
| 402 | 1^{st} layer |
| 404 | 2^{nd} layer |
| 406 | area for left foot |
| 408 | area for right foot |
| Ch1 . . . ChN | first channel . . . Nth channel |
| S1 . . . SN | first section . . . Nth section |

### Glossary

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

This application is filed with an appendix, herein incorporated by reference in its entirety. Unless aspects of the appendix are expressly included in the claims, nothing in the appendix shall limit the present disclosure.

What is claimed is:

1. A system for monitoring movements comprising:
an elastomeric pressure sensor patterned with a plurality of sensing channels, the plurality of sensing channels being formed with a plurality of microchannels filled with:
(a) a conductive liquid; and
(b) a substrate material; and
a plurality of sections included within each of the plurality of channels;
wherein each of the plurality of sections are distinguishable from one another because of a difference in the number of microchannels included therein.

2. The system of claim 1, wherein the number of microchannels linearly increase from a first side of a first sensing channel selected from the plurality of sensing channels to a second side of the first sensing channel selected from the plurality of sensing channels.

3. The system of claim 2, wherein the number of microchannels linearly decrease from a first side of a second sensing channel selected from the plurality of sensing channels to a second side of the second sensing channel selected from the plurality of sensing channels.

4. The system of claim 1, wherein:
the plurality of microchannels comprise two microchannels; and
the plurality of sections comprise five sections.

5. The system of claim 1, wherein the conductive liquid comprises Eutectic gallium-indium (EGaIn).

6. The system of claim 1, wherein the synthetic elastomer comprises a silicone mixture.

7. The system of claim 1, wherein the plurality of microchannels further comprise air.

8. The system of claim 1, wherein the elastomeric pressure sensor (i) is included within a wearable object; (ii) implemented into a mat, (iii) forms part of a controller, or (iv) attached to a movable object.

9. The system of claim 1, further comprising another sensor that collects biometric data that relates to an aspect other than pressure.

10. A method for monitoring a movement, the method comprising:
analyzing a widthwise and lengthwise distribution in a plurality of sensing channels having a plurality of sections that are distinguishable from one another because of a difference in the number of microchannels included therein;
wherein the microchannels comprise:
(a) a conductive liquid; and
(b) a substrate material.

11. The method of claim 10, further comprising monitoring human gait with said analysis.

12. The method of claim 10, further comprising attaching an elastomeric pressure sensor that includes said sensing channels to an object that experiences the movement.

13. The method of claim 10, further comprising allowing the object that experiences the movement to apply repeated, patterned pressure to the plurality of sensing channels.

14. A multi-sectioned elastomeric pressure sensor comprising:
a plurality of sensing channels, the plurality of sensing channels being formed with a plurality of microchannels comprise:
(a) a conductive liquid; and
(b) a substrate material; and
wherein a plurality of sections in the multi-sectioned elastomeric pressure sensor are distinguishable from one another because of a difference in the number of microchannels included therein.

15. The multi-sectioned elastomeric pressure sensor of claim 14, wherein the number of microchannels linearly increase from a first side of a first sensing channel selected from the plurality of sensing channels to a second side of the first sensing channel selected from the plurality of sensing channels.

16. The multi-section elastomeric pressure sensor of claim 15, wherein the number of microchannels linearly decrease from a first side of a second sensing channel selected from the plurality of sensing channels to a second side of the second sensing channel selected from the plurality of sensing channels.

17. The multi-sectioned elastomeric pressure sensor of claim 14, wherein the plurality of microchannels comprise two microchannels and five sections.

18. The multi-sectioned elastomeric pressure sensor of claim 14, wherein the conductive liquid comprises a liquid metal.

19. The multi-sectioned elastomeric pressure sensor of claim 14, wherein the synthetic elastomer comprises a synthetic elastomer.

20. The multi-sectioned elastomeric pressure sensor of claim 14, wherein the plurality of microchannels further comprise air.
